(19) United States
(12) Patent Application Publication
(10) Pub. No.: US 2025/0075269 A1
(43) Pub. Date: Mar. 6, 2025

(54) COMPOSITIONS AND METHODS FOR NUCLEIC ACID SEQUENCING

(71) Applicant: Illumina, Inc., San Diego, CA (US)

(72) Inventors: Xiaolin Wu, Cambridge (GB); Madushani Dharmarwardana, Cambridge (GB); Harry John Meats, Cambridge (GB)

(21) Appl. No.: 18/820,008

(22) Filed: Aug. 29, 2024

Related U.S. Application Data
(60) Provisional application No. 63/579,897, filed on Aug. 31, 2023.

(51) Int. Cl. C12Q 1/6869; C12Q 1/6806; C12Q 1/6876; G01N 33/58

(52) U.S. Cl. CPC C12Q 1/6869; C12Q 1/6806; C12Q 1/6876; G01N 33/582

(57) ABSTRACT

Embodiments of the present disclosure relate to methods and kits for sequencing by synthesis utilizing biorthogonal chemical reactions between unlabeled nucleotides and post incorporation labeling reagents.

### FIELD

The present disclosure generally relates to compositions, kits, methods and systems for nucleic acid sequencing applications.

### BACKGROUND

Nucleic acid sequencing methodology has evolved significantly from the chemical degradation methods used by Maxam and Gilbert and the strand elongation methods used by Sanger. Today several sequencing methodologies are in use which allow for the parallel processing of thousands of nucleic acids all in a single sequencing run. The instrumentation that performs such methods is typically large and expensive since the current methods typically rely on large amounts of expensive reagents and multiple sets of optic filters to record nucleic acid incorporation into sequencing reactions.

It has become clear that the need for high-throughput, smaller, less expensive DNA sequencing technologies will be beneficial for reaping the rewards of genome sequencing. Personalized healthcare is moving toward the forefront and will benefit from such technologies. The sequencing of an individual's genome to identify potential mutations and abnormalities will be crucial in identifying if a person has a particular disease, followed by subsequent therapies tailored to that individual. To accommodate such endeavor, sequencing technologies should not only have high throughput capabilities, but also have scalability. As such, there exists a need for new sequencing methods that improve on speed, error read, and are also cost effective.

### SUMMARY

One aspect of the present disclosure relates to a method of determining the sequences of a plurality of different target polynucleotides, comprising:
(a) contacting a solid support with a solution comprising sequencing primers under hybridization conditions, wherein the solid support comprises a plurality of different target polynucleotides immobilized thereon; and the sequencing primers are complementary to at least a portion of the target polynucleotides;
(b) contacting the solid support with an incorporation mixture comprising DNA polymerase and one more of four different types of nucleotides under conditions suitable for DNA polymerase-mediated primer extension, and incorporating one type of nucleotides into the sequencing primers to produce extended copy polynucleotides; wherein
   each of the four types of nucleotides comprises a 3′ blocking group; and
   the incorporation mixture comprises a first type of unlabeled nucleotide having a first functional moiety covalently attached to the first type of unlabeled nucleotide;
(c) contacting the extended copy polynucleotides with a first labeling reagent comprising a first detectable label and a first reactive moiety that reacts specifically with the first functional moiety of the first type of unlabeled nucleotides to form covalent bonding;
(d) imaging the solid support and performing one or more fluorescent measurements of the extended copy polynucleotides; and
(e) removing the 3′ blocking group of the incorporated nucleotides.

In some embodiments of the method, the incorporation mixture comprises the first type of unlabeled nucleotide having the first functional moiety covalently attached to the first type of unlabeled nucleotide as described herein;
a second type of nucleotide;
a third type of nucleotide; and
a fourth type of unlabeled nucleotide;
wherein the fourth type of unlabeled nucleotide is not capable of reacting with the first labeling reagent. In some such embodiments, each of the second type of nucleotide and the third type of nucleotide is labeled. In some other embodiments, the third type of nucleotide is labeled, and the second type of nucleotide is a second type of unlabeled nucleotide having a second functional moiety covalently attached to the second type of unlabeled nucleotide. In still other embodiments, the second type of nucleotide is a second type of unlabeled nucleotide having a second functional moiety covalently attached to the second type of unlabeled nucleotide, and the third type of nucleotide is a mixture of a third type of unlabeled nucleotide having a first functional moiety covalently attached to the third type of unlabeled nucleotide and a third type of unlabeled nucleotide having a second functional moiety covalently attached to the third type of unlabeled nucleotide In some such embodiments, step (c) of the method further comprises contacting the extended copy polynucleotides with a second labeling reagent comprising a second detectable label and a second reactive moiety that is capable of reacting specifically with the second functional moiety to form covalent bonding, and wherein the fourth type of unlabeled nucleotide is not capable of reacting with either the first labeling reagent or the second labeling reagent. In some other embodiments, the third type of nucleotide is a third type of unlabeled nucleotide having a third functional moiety covalently attached to the third type of unlabeled nucleotide, and step (c) of the method further comprises contacting the extended copy polynucleotides with a third labeling reagent comprising a third detectable label and a third reactive moiety that is capable of reacting specifically with the third functional moiety of the third type of unlabeled nucleotide to form covalent bonding, and wherein the fourth type of unlabeled nucleotide is not capable of reacting with any one of the first labeling reagent, the second labeling reagent, or the third labeling reagent.

Another aspect of the present disclosure relates to a kit for sequencing application, comprising:
an incorporation mixture composition comprising one or more of four different types of nucleotides each comprising a 3′ blocking group, wherein a first type of unlabeled nucleotide having a first functional moiety covalently attached to the first type of unlabeled nucleotide; and
a first labeling reagent comprising a first detectable label and a first reactive moiety that is capable of reacting specifically with the first functional moiety of the first type of unlabeled nucleotides to form covalent bonding.

In some further embodiments, the kit comprises:
the first type of unlabeled nucleotide having the first functional moiety covalently attached to the first type of unlabeled nucleotide as described herein;
a second type of nucleotide;
a third type of nucleotide;
a fourth type of unlabeled nucleotide; and
the first labeling reagent comprising a reactive moiety that is capable of reacting specifically with the first functional moiety of the first type of unlabeled nucleotides to form covalent bonding;
wherein the fourth type of unlabeled nucleotide is not capable of reacting with the first labeling reagent. In some such embodiments, each of the second type of nucleotide and the third type of nucleotide is labeled. In some other embodiments, the second type of nucleotide is a second type of unlabeled nucleotide having a second functional moiety covalently attached to the second type of unlabeled nucleotide, and the kit further comprises a second labeling reagent comprising a second detectable label and a second reactive moiety that is capable of reacting specifically with the second functional moiety to form covalent bonding, and wherein the fourth type of unlabeled nucleotide is not capable of reacting with either the first labeling reagent or the second labeling reagent. In some such embodiments, the third type of nucleotide is a mixture of a third type of unlabeled nucleotide having a first functional moiety covalently attached to the third type of unlabeled nucleotide and a third type of unlabeled nucleotide having a second functional moiety covalently attached to the third type of unlabeled nucleotide. In some other embodiments, the third type of nucleotide is a third type of unlabeled nucleotide having a third functional moiety covalently attached to the third type of unlabeled nucleotide, and the kit further comprises a third labeling reagent comprising a third detectable label and a third reactive moiety that is capable of reacting specifically with the third functional moiety of the third type of unlabeled nucleotide to form covalent bonding, and wherein the fourth type of unlabeled nucleotide is not capable of reacting with any one of the first labeling reagent, the second labeling reagent, or the third labeling reagent.

### BRIEF DESCRIPTION OF THE DRAWINGS

FIG. 1A illustrates a general scheme of bio-orthogonal post incorporation labeling method in sequencing by synthesis (SBS), according to certain embodiments of the present disclosure.

FIG. 1B illustrates an example of bio-orthogonal post incorporation labeling method described in FIG. 1A in which the detectable label is covalently attached to the nucleobase of the incorporated nucleotide, according to certain embodiments of the present disclosure.

FIG. 1C illustrates an example of bio-orthogonal post incorporation labeling method described in FIG. 1A in which the detectable label is covalently attached to the 3′ position of the 2′-deoxyribose of the incorporated nucleotide, according to certain embodiments of the present disclosure.

FIG. 2A is a scatterplot of a blue/green two-channel SBS run conducted with an incorporation mixture containing a set of nucleotides including an unlabeled C nucleotide with azido moiety, and a post incorporation labeling mixture containing a blue coumarin dye A1 functionalized with a dibenzocyclooctyne (DBCO) moiety.

FIG. 2B is a line chart illustrating normalized C nucleotide signal as a function of the concentration of a post incorporation labeling mixture containing a blue coumarin dye (A1-DBCO).

FIG. 2C is a bar chart illustrating the normalized percentage (%) brightness of C nucleotide signal of an incorporation mixture containing a labeled fully functionalized C nucleotide (ffC), as compared to an incorporation mixture containing unlabeled C nucleotide with azido moiety, and a post incorporation labeling mixture containing a blue coumarin dye A1-DBCO at various concentrations.

FIG. 3A is a scatterplot of a blue/green two-channel SBS run conducted with a standard incorporation mixture containing fully labeled nucleotides in which the ffC is labeled with a blue coumarin dye A.

FIG. 3B is a scatterplot of a blue/green two-channel SBS run conducted with an incorporation mixture containing a set of nucleotides including an unlabeled C nucleotide with trans-cyclooctene (TCO) moiety, and a post incorporation labeling mixture containing a blue coumarin dye A1 functionalized with a tetrazine (Tz) moiety.

FIG. 3C is a line chart illustrating normalized C nucleotide signal as a function of the concentration of a blue coumarin dye (A1-Tz) in the post incorporation labeling mixture, as compared to the C nucleotide signal of an incorporation mixture containing ffC labeled with coumarin dye A.

FIG. 4A is a scatterplot of a blue/green two-channel SBS run conducted with an incorporation mixture containing a set of nucleotides including an unlabeled C nucleotide with a DBCO moiety covalently attached to the nucleotide through a cleavable linker, and a post incorporation labeling mixture containing a blue coumarin dye A1 functionalized with azido moiety.

FIG. 4B is a scatterplot of a blue/green two-channel SBS run conducted with an incorporation mixture containing a set of nucleotides including an unlabeled C nucleotide with a DBCO moiety covalently attached to the nucleotide through a cleavable linker with extra polyethylene glycol (PEG) spacer, and a post incorporation labeling mixture containing a blue coumarin dye A1 functionalized with an azido moiety.

FIG. 5A is a scatterplot of a blue/green two-channel SBS run conducted with an incorporation mixture containing a set of nucleotides including an unlabeled C nucleotide with a TCO moiety, and a post incorporation labeling mixture containing a blue coumarin dye A1 functionalized with a Tz moiety, according to an embodiment of the present disclosure. The post incorporation labeling reagent was introduced to the flowcell surface directly after the incorporation mixture was introduced.

FIG. 5B is a scatterplot of a blue/green two-channel SBS run conducted with the same incorporation mixture and the post-incorporation labeling mixture as described in FIG. 5A, in which a high salt buffer was used to wash the flowcell surface after the incorporation mixture, and also after the post incorporation labeling step.

FIG. 6A is a scatterplot of a blue/green two-channel SBS run conducted with a standard incorporation mixture containing fully labeled nucleotides in which the ffC is labeled with a blue coumarin dye A.

FIG. 6B is a scatterplot of a blue/green two-channel SBS run conducted with an incorporation mixture containing a set of nucleotides including an unlabeled C nucleotide with a TCO moiety covalently attached to the nucleotide through a cleavable linker with one cleavage site, and a post-incorporation labeling mixture containing a blue dye A1 functionalized with a Tz moiety.

FIG. 6C is a scatterplot of a blue/green two-channel SBS run conducted with an incorporation mixture containing a set of nucleotides including an unlabeled C nucleotide with a TCO moiety covalently attached to the nucleotide through a cleavable linker with two cleavage sites, and a post-incorporation labeling mixture containing a blue coumarin dye A1 functionalized with a Tz moiety.

FIG. 7A is a scatterplot of a blue/green two-channel SBS run conducted with a standard incorporation mixture containing fully labeled nucleotides in which the ffC is labeled with a blue coumarin dye A.

FIG. 7B is a scatterplot of a blue/green two-channel SBS run conducted with an incorporation mixture containing a set of nucleotides including an unlabeled A nucleotide with a TCO moiety and an unlabeled T nucleotide with a DBCO moiety, and a set of post-incorporation labeling mixture containing a blue coumarin dye A1 functionalized with an azido moiety, and a green dye NR500C4 functionalized with a pyridyl-tetrazine moiety (Py-Tz). The post-labeling reagents were flushed through the flowcell surface after the incorporation step.

FIG. 7C is a scatterplot of a blue/green two-channel SBS run conducted with the same incorporation mixture as described in FIG. 7B. The post-labeling mixture was incubated on the flowcell surface after the incorporation step at 60° C. for about 20 s.

### DETAILED DESCRIPTION

The present disclosure provides next-generation sequencing kits, methods, systems and compositions. Certain disclosure relates to methods, kits and compositions for two-channel nucleic acid sequencing applications using blue and green light excitations (e.g., lasers at 450-460 nm and 520-540 nm).

### Definitions

Unless defined otherwise, all technical and scientific terms used herein have the same meaning as is commonly understood by one of ordinary skill in the art. The use of the term “including” as well as other forms, such as “include”, “includes,” and “included,” is not limiting. The use of the term “having” as well as other forms, such as “have”, “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps but may include additional steps. When used in the context of a compound, composition, or device, the term “comprising” means that the compound, composition, or device includes at least the recited features or components, but may also include additional features or components.

As used herein, common organic abbreviations are defined as follows:
° C. Temperature in degrees Centigrade
dATP Deoxyadenosine triphosphate
dCTP Deoxycytidine triphosphate
dGTP Deoxyguanosine triphosphate
dTTP Deoxythymidine triphosphate
ddNTP Dideoxynucleotide triphosphate
ffA Fully functionalized A nucleotide
ffC Fully functionalized C nucleotide
ffG Fully functionalized G nucleotide
ffN Fully functionalized nucleotide
ffT Fully functionalized T nucleotide
LED Light emitting diode
SBS Sequencing by synthesis

As used herein, the term “array” refers to a population of different probe molecules that are attached to one or more substrates such that the different probe molecules can be differentiated from each other according to relative location. An array can include different probe molecules that are each located at a different addressable location on a substrate. Alternatively, or additionally, an array can include separate substrates each bearing a different probe molecule, wherein the different probe molecules can be identified according to the locations of the substrates on a surface to which the substrates are attached or according to the locations of the substrates in a liquid. Exemplary arrays in which separate substrates are located on a surface include, without limitation, those including beads in wells as described, for example, in U.S. Pat. No. 6,355,431 B1, US 2002/0102578 and PCT Publication No. WO 00/63437. Exemplary formats that can be used in the invention to distinguish beads in a liquid array, for example, using a microfluidic device, such as a fluorescent activated cell sorter (FACS), are described, for example, in U.S. Pat. No. 6,524,793. Further examples of arrays that can be used in the invention include, without limitation, those described in U.S. Pat. Nos. 5,429,807; 5,436,327; 5,561,071; 5,583,211; 5,658,734; 5,837,858; 5,874,219; 5,919,523; 6,136,269; 6,287,768; 6,287,776; 6,288,220; 6,297,006; 6,291,193; 6,346,413; 6,416,949; 6,482,591; 6,514,751 and 6,610,482; and WO 93/17126; WO 95/11995; WO 95/35505; EP 742 287; and EP 799 897.

As used herein, the term “covalently attached” or “covalently bonded” refers to the forming of a chemical bonding that is characterized by the sharing of pairs of electrons between atoms. For example, a covalently attached polymer coating refers to a polymer coating that forms chemical bonds with a functionalized surface of a substrate, as compared to attachment to the surface via other means, for example, adhesion or electrostatic interaction. It will be appreciated that polymers that are attached covalently to a surface can also be bonded via means in addition to covalent attachment.

As used herein, the term “non-covalent interactions” differs from a covalent bond in that it does not involve the sharing of electrons, but rather involves more dispersed variations of electromagnetic interactions between molecules or within a molecule. Non-covalent interactions can be generally classified into four categories, electrostatic, π-effects, van der Waals forces, and hydrophobic effects. Non-limiting examples of electrostatic interactions include ionic interactions, hydrogen bonding (a specific type of dipole-dipole interaction), halogen bonding, etc. Van der Walls forces are a subset of electrostatic interaction involving permanent or induced dipoles or multipoles. π-effects can be broken down into numerous categories, including (but not limited to) π-π interactions, cation-π & anion-π interactions, and polar-π interactions. In general, π-effects are associated with the interactions of molecules with the π-orbitals of a molecular system, such as benzene. The hydrophobic effect is the tendency of nonpolar substances to aggregate in aqueous solution and exclude water molecules. Non-covalent interactions can be both intermolecular and intramolecular. Non-covalent interactions can be both intermolecular and intramolecular.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene” or “alkenylene.”

The term “halogen” or “halo,” as used herein, means any one of the radio-stable atoms of column 7 of the Periodic Table of the Elements, e.g., fluorine, chlorine, bromine, or iodine, with fluorine and chlorine being preferred.

As used herein, “Cₐ to C_{b},” “Cₐ-C_{b},” or “C_{a-b}” in which “a” and “b” are integers refer to the number of carbon atoms in an alkyl, alkenyl or alkynyl group, or the number of ring atoms of a cycloalkyl or aryl group. That is, the alkyl, the alkenyl, the alkynyl, the ring of the cycloalkyl, and ring of the aryl can contain from “a” to “b,” inclusive, carbon atoms. For example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—; a C₃ to C₄ cycloalkyl group refers to all cycloalkyl groups having from 3 to 4 carbon atoms, that is, cyclopropyl and cyclobutyl. Similarly, a “4 to 6 membered heterocyclyl” group refers to all heterocyclyl groups with 4 to 6 total ring atoms, for example, azetidine, oxctane, oxazoline, pyrrolidine, piperidine, piperazine, morpholine, and the like. If no “a” and “b” are designated with regard to an alkyl, alkenyl, alkynyl, cycloalkyl, or aryl group, the broadest range described in these definitions is to be assumed. As used herein, the term “C₁-C₆” includes C₁, C₂, C₃, C₄, C₅ and C₆, and a range defined by any of the two numbers For example, C₁-C₆ alkyl includes C₁, C₂, C₃, C₄, C₅ and C₆ alkyl, C₂-C₆ alkyl, C₁-C₃ alkyl, etc. Similarly, C₂-C₆ alkenyl includes C₂, C₃, C₄, C₅ and C₆ alkenyl, C₂-C₅ alkenyl, C₃-C₄ alkenyl, etc.; and C₂-C₆ alkynyl includes C₂, C₃, C₄, C₅ and C₆ alkynyl, C₂-C₅ alkynyl, C₃-C₄ alkynyl, etc. C₃-C₈ cycloalkyl each includes hydrocarbon ring containing 3, 4, 5, 6, 7 and 8 carbon atoms, or a range defined by any of the two numbers, such as C₃-C₇ cycloalkyl or C₅-C₆ cycloalkyl.

As used herein, “alkyl” refers to a straight or branched hydrocarbon chain that is fully saturated (i.e., contains no double or triple bonds). The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 6 carbon atoms. The alkyl group may be designated as “C₁-C₄ alkyl” or similar designations. By way of example only, “C₁-C₆ alkyl” indicates that there are one to six carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

As used herein, “alkoxy” refers to the formula-OR wherein R is an alkyl as is defined above, such as “C₁-C₉ alkoxy,” including but not limited to methoxy, ethoxy, n-propoxy, 1-methylethoxy (isopropoxy), n-butoxy, iso-butoxy, sec-butoxy, and tert-butoxy, and the like.

As used herein, “alkenyl” refers to a straight or branched hydrocarbon chain containing one or more double bonds. The alkenyl group may have 2 to 20 carbon atoms, although the present definition also covers the occurrence of the term “alkenyl” where no numerical range is designated. The alkenyl group may also be a medium size alkenyl having 2 to 9 carbon atoms. The alkenyl group could also be a lower alkenyl having 2 to 6 carbon atoms. The alkenyl group may be designated as “C₂-C₆ alkenyl” or similar designations. By way of example only, “C₂-C₆ alkenyl” indicates that there are two to six carbon atoms in the alkenyl chain, i.e., the alkenyl chain is selected from the group consisting of ethenyl, propen-1-yl, propen-2-yl, propen-3-yl, buten-1-yl, buten-2-yl, buten-3-yl, buten-4-yl, 1-methyl-propen-1-yl, 2-methyl-propen-1-yl, 1-ethyl-ethen-1-yl, 2-methyl-propen-3-yl, buta-1,3-dienyl, buta-1,2,-dienyl, and buta-1,2-dien-4-yl. Typical alkenyl groups include, but are in no way limited to, ethenyl, propenyl, butenyl, pentenyl, and hexenyl, and the like.

The term “aromatic” refers to a ring or ring system having a conjugated pi electron system and includes both carbocyclic aromatic (e.g., phenyl) and heterocyclic aromatic groups (e.g., pyridine). The term includes monocyclic or fused-ring polycyclic (i.e., rings which share adjacent pairs of atoms) groups provided that the entire ring system is aromatic.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms, although the present definition also covers the occurrence of the term “aryl” where no numerical range is designated. In some embodiments, the aryl group has 6 to 10 carbon atoms. The aryl group may be designated as “C₆-C₁₀ aryl,” “C₆ or C₁₀ aryl,” or similar designations. Examples of aryl groups include, but are not limited to, phenyl, naphthyl, azulenyl, and anthracenyl.

An “aralkyl” or “arylalkyl” is an aryl group connected, as a substituent, via an alkylene group, such as “C₇₋₁₄ aralkyl” and the like, including but not limited to benzyl, 2-phenylethyl, 3-phenylpropyl, and naphthylalkyl. In some cases, the alkylene group is a lower alkylene group (i.e., a C₁-C₆ alkylene group).

As used herein, “aryloxy” refers to RO— in which R is an aryl, as defined above, such as but not limited to phenyl.

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. In some embodiments, the heteroaryl group has 5 to 10 ring members or 5 to 7 ring members. The heteroaryl group may be designated as “5-7 membered heteroaryl,” “5-10 membered heteroaryl,” or similar designations. Examples of heteroaryl rings include, but are not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, triazinyl, quinolinyl, isoquinolinyl, benzoimidazolyl, benzoxazolyl, benzothiazolyl, indolyl, isoindolyl, and benzothienyl.

A “heteroaralkyl” or “heteroarylalkyl” is heteroaryl group connected, as a substituent, via an alkylene group. Examples include but are not limited to 2-thienylmethyl, 3-thienylmethyl, furylmethyl, thienylethyl, pyrrolylalkyl, pyridylalkyl, isoxazollylalkyl, and imidazolylalkyl. In some cases, the alkylene group is a lower alkylene group (i.e., a C₁-C₆ alkylene group).

As used herein, “carbocyclyl” means a non-aromatic cyclic ring or ring system containing only carbon atoms in the ring system backbone. When the carbocyclyl is a ring system, two or more rings may be joined together in a fused, bridged or spiro-connected fashion. Carbocyclyls may have any degree of saturation provided that at least one ring in a ring system is not aromatic. Thus, carbocyclyls include cycloalkyls, cycloalkenyls, and cycloalkynyls. The carbocyclyl group may have 3 to 20 carbon atoms, although the present definition also covers the occurrence of the term “carbocyclyl” where no numerical range is designated. The carbocyclyl group may also be a medium size carbocyclyl having 3 to 10 carbon atoms. The carbocyclyl group could also be a carbocyclyl having 3 to 6 carbon atoms. The carbocyclyl group may be designated as “C₃-C₆ carbocyclyl” or similar designations. Examples of carbocyclyl rings include, but are not limited to, cyclopropyl, cyclobutyl, cyclopentyl, cyclohexyl, cyclohexenyl, 2,3-dihydro-indene, bicycle[2.2.2]octanyl, adamantyl, and spiro[4.4]nonanyl.

As used herein, “cycloalkyl” means a fully saturated carbocyclyl ring or ring system. Examples include cyclopropyl, cyclobutyl, cyclopentyl, and cyclohexyl.

As used herein, “heterocyclyl” means a non-aromatic cyclic ring or ring system containing at least one heteroatom in the ring backbone. Heterocyclyls may be joined together in a fused, bridged or spiro-connected fashion. Heterocyclyls may have any degree of saturation provided that at least one ring in the ring system is not aromatic. The heteroatom(s) may be present in either a non-aromatic or aromatic ring in the ring system. The heterocyclyl group may have 3 to 20 ring members (i.e., the number of atoms making up the ring backbone, including carbon atoms and heteroatoms), although the present definition also covers the occurrence of the term “heterocyclyl” where no numerical range is designated. The heterocyclyl group may also be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations. In preferred six membered monocyclic heterocyclyls, the heteroatom(s) are selected from one up to three of O, N or S, and in preferred five membered monocyclic heterocyclyls, the heteroatom(s) are selected from one or two heteroatoms selected from O, N, or S. Examples of heterocyclyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline.

As used herein, “(aryl)alkyl” refer to an aryl group, as defined above, connected, as a substituent, via an alkylene group, as described above. The alkylene and aryl group of an aralkyl may be substituted or unsubstituted. Examples include but are not limited to benzyl, 2-phenylalkyl, 3-phenylalkyl, and naphthylalkyl. In some embodiments, the alkylene is an unsubstituted straight chain containing 1, 2, 3, 4, 5, or 6 methylene unit(s).

As used herein, “(heteroaryl)alkyl” refer to a heteroaryl group, as defined above, connected, as a substituent, via an alkylene group, as defined above. The alkylene and heteroaryl group of heteroaralkyl may be substituted or unsubstituted. Examples include but are not limited to 2-thienylalkyl, 3-thienylalkyl, furylalkyl, thienylalkyl, pyrrolylalkyl, pyridylalkyl, isoxazolylalkyl, and imidazolylalkyl, and their benzo-fused analogs. In some embodiments, the alkylene is an unsubstituted straight chain containing 1, 2, 3, 4, 5, or 6 methylene unit(s).

As used herein, “(heterocyclyl)alkyl” refer to a heterocyclic or a heterocyclyl group, as defined above, connected, as a substituent, via an alkylene group, as defined above. The alkylene and heterocyclyl groups of a (heterocyclyl)alkyl may be substituted or unsubstituted. Examples include but are not limited to (tetrahydro-2H-pyran-4-yl)methyl, (piperidin-4-yl)ethyl, (piperidin-4-yl) propyl, (tetrahydro-2H-thiopyran-4-yl)methyl, and (1,3-thiazinan-4-yl)methyl. In some embodiments, the alkylene is an unsubstituted straight chain containing 1, 2, 3, 4, 5, or 6 methylene unit(s).

As used herein, “(carbocyclyl)alkyl” refer to a carbocyclyl group (as defined herein) connected, as a substituent, via an alkylene group. Examples include but are not limited to cyclopropylmethyl, cyclobutylmethyl, cyclopentylethyl, and cyclohexylpropyl. In some embodiments, the alkylene is an unsubstituted straight chain containing 1, 2, 3, 4, 5, or 6 methylene unit(s).

As used herein, “alkoxyalkyl” or “(alkoxy)alkyl” refers to an alkoxy group connected via an alkylene group, such as C₂-C₈ alkoxyalkyl, or (C₁-C₆ alkoxy) C₁-C₆ alkyl, for example, (CH₂)₁₋₃—OCH₃.

As used herein, “—O-alkoxyalkyl” or “—O-(alkoxy)alkyl” refers to an alkoxy group connected via an —O-(alkylene) group, such as —O—(C₁-C₆ alkoxy) C₁-C₆ alkyl, for example, —O—(CH₂)₁₋₃—OCH₃.

As used herein, “haloalkyl” refers to an alkyl group in which one or more of the hydrogen atoms are replaced by a halogen (e.g., mono-haloalkyl, di-haloalkyl, and tri-haloalkyl). Such groups include but are not limited to, chloromethyl, fluoromethyl, difluoromethyl, trifluoromethyl and 1-chloro-2-fluoromethyl, 2-fluoroisobutyl. A haloalkyl may be substituted or unsubstituted.

As used herein, “haloalkoxy” refers to an alkoxy group in which one or more of the hydrogen atoms are replaced by a halogen (e.g., mono-haloalkoxy, di-haloalkoxy and tri-haloalkoxy). Such groups include but are not limited to, chloromethoxy, fluoromethoxy, difluoromethoxy, trifluoromethoxy and 1-chloro-2-fluoromethoxy, 2-fluoroisobutoxy. A haloalkoxy may be substituted or unsubstituted.

An “amino” group refers to a —NH₂ group. The term “mono-substituted amino group” as used herein refers to an amino (—NH₂) group where one of the hydrogen atom is replaced by a substituent. The term “di-substituted amino group” as used herein refers to an amino (—NH₂) group where each of the two hydrogen atoms is replaced by a substituent. The term “optionally substituted amino,” as used herein refer to a —NR_{A}R_{B} group where R_{A} and R_{B} are independently hydrogen, alkyl, cycloalkyl, aryl, heteroaryl, heterocyclyl, aralkyl, or heterocyclyl(alkyl), as defined herein.

An “O-carboxy” group refers to a “—OC(═O) R” group in which R is selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

A “C-carboxy” group refers to a “—C(═O) OR” group in which R is selected from the group consisting of hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein. A non-limiting example includes carboxyl (i.e., —C(═O) OH).

A “sulfonyl” group refers to an “—SO₂R” group in which R is selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

A “sulfino” group refers to a “—S(═O) OH” group.

A “sulfo” group refers to a “—S(═O)₂OH” or “—SO₃H” group.

A “sulfonate” group refers to a “—SO₃” group.

A “sulfate” group refers to “—SO₄” group.

A “S-sulfonamido” group refers to a “—SO₂NR_{A}R_{B}” group in which R_{A} and R_{B} are each independently selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

An “N-sulfonamido” group refers to a “—N(R_{A}) SO₂R_{B}” group in which R_{A} and R_{b} are each independently selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

A “C-amido” group refers to a “—C(═O) NR_{A}R_{B}” group in which R_{A} and R_{B} are each independently selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

An “N-amido” group refers to a “—N(R_{A})C(═O)R_{B}” group in which R_{A} and R_{B} are each independently selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

An “O-carbamyl” group refers to a “—OC(═O)N(R_{A}R_{B})” group in which R_{A} and R_{B} can be the same as defined with respect to S-sulfonamido. An O-carbamyl may be substituted or unsubstituted.

An “N-carbamyl” group refers to an “ROC(═O)N(R_{A})—” group in which R and R_{A} can be the same as defined with respect to N-sulfonamido. An N-carbamyl may be substituted or unsubstituted.

An “O-thiocarbamyl” group refers to a “—OC(═S)—N(R_{A}R_{B})” group in which R_{A} and R_{B} can be the same as defined with respect to S-sulfonamido. An O-thiocarbamyl may be substituted or unsubstituted.

An “N-thiocarbamyl” group refers to an “ROC(═S)N(R_{A})—” group in which R and R_{A} can be the same as defined with respect to N-sulfonamido. An N-thiocarbamyl may be substituted or unsubstituted.

The term “alkylamino” or “(alkyl)amino” refers to an amino group wherein one or both hydrogen is replaced by an alkyl group.

An “(alkoxy)alkyl” group refers to an alkoxy group connected via an alkylene group, such as a “(C₁-C₆ alkoxy) C₁-C₆ alkyl” and the like.

The term “hydroxy” as used herein refers to a —OH group.

The term “cyano” group as used herein refers to a “—CN” group.

The term “azido” as used herein refers to a —N₃ group.

The term “isonitrile” as used herein refers to a “—N⁺≡C⁻” group.

When a group is described as “optionally substituted” it may be either unsubstituted or substituted. Likewise, when a group is described as being “substituted,” the substituent may be selected from one or more of the indicated substituents. As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more substituents independently selected from C₁-C₆ alkyl, C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 3-10 membered heterocyclyl (optionally substituted with halo, C₁-C₆ alkyl, C₁-C₆ alkoxy, C₁-C₆ haloalkyl, and C₁-C₆ haloalkoxy), 3-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), (aryl) C₁-C₆ alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), (5-10 membered heteroaryl) C₁-C₆ alkyl (optionally substituted with halo, C₁-C₆ alkyl, C₁-C₆ alkoxy, C₁-C₆ haloalkyl, and C₁-C₆ haloalkoxy), halo, —CN, hydroxy, C₁-C₆ alkoxy, (C₁-C₆ alkoxy) C₁-C₆ alkyl, —O(C₁-C₆ alkoxy) C₁-C₆ alkyl; (C₁-C₆ haloalkoxy) C₁-C₆ alkyl; —O(C₁-C₆ haloalkoxy) C₁-C₆ alkyl; aryloxy, sulfhydryl (mercapto), halo(C₁-C₆)alkyl (e.g., —CF₃), halo(C₁-C₆)alkoxy (e.g., —OCF₃), C₁-C₆ alkylthio, arylthio, amino, amino (C₁-C₆)alkyl, nitro, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, —SO₃H, sulfonate, sulfate, sulfino, —OSO₂C₁₋₄alkyl, monophosphate, diphosphate, triphosphate, and oxo (═O). Wherever a group is described as “optionally substituted” that group can be substituted with the above substituents.

In each instance where a single mesomeric form of a compound described herein is shown, the alternative mesomeric forms are equally contemplated.

As used herein, a “nucleotide” includes a nitrogen containing heterocyclic base, a sugar, and one or more phosphate groups. They are monomeric units of a nucleic acid sequence. In RNA, the sugar is a ribose, and in DNA a deoxyribose, i.e., a sugar lacking a hydroxyl group that is present in ribose. The nitrogen containing heterocyclic base can be purine, deazapurine, or pyrimidine base. Purine bases include adenine (A) and guanine (G), and modified derivatives or analogs thereof, such as 7-deaza adenine or 7-deaza guanine. Pyrimidine bases include cytosine (C), thymine (T), and uracil (U), and modified derivatives or analogs thereof. The C-1 atom of deoxyribose is bonded to N-1 of a pyrimidine or N-9 of a purine.

As used herein, a “nucleotide conjugate” generally refers to a nucleotide labeled with a fluorescent moiety, optionally through a cleavage linker as described herein. In some embodiment, when a nucleotide conjugate is described as an unlabeled nucleotide, such nucleotide does not include a fluorescent moiety. In some further embodiments, an unlabeled nucleotide conjugate also does not have a cleavable linker.

As used herein, a “nucleoside” is structurally similar to a nucleotide but is missing the phosphate moieties. An example of a nucleoside analogue would be one in which the label is linked to the base and there is no phosphate group attached to the sugar molecule. The term “nucleoside” is used herein in its ordinary sense as understood by those skilled in the art. Examples include, but are not limited to, a ribonucleoside comprising a ribose moiety and a deoxyribonucleoside comprising a deoxyribose moiety. A modified pentose moiety is a pentose moiety in which an oxygen atom has been replaced with a carbon and/or a carbon has been replaced with a sulfur or an oxygen atom. A “nucleoside” is a monomer that can have a substituted base and/or sugar moiety. Additionally, a nucleoside can be incorporated into larger DNA and/or RNA polymers and oligomers.

The term “purine base” is used herein in its ordinary sense as understood by those skilled in the art and includes its tautomers. Similarly, the term “pyrimidine base” is used herein in its ordinary sense as understood by those skilled in the art and includes its tautomers. A non-limiting list of optionally substituted purine-bases includes purine, adenine, guanine, deazapurine, 7-deaza adenine, 7-deaza guanine. hypoxanthine, xanthine, alloxanthine, 7-alkylguanine (e.g., 7-methylguanine), theobromine, caffeine, uric acid and isoguanine. Examples of pyrimidine bases include, but are not limited to, cytosine, thymine, uracil, 5,6-dihydrouracil and 5-alkylcytosine (e.g., 5-methylcytosine).

As used herein, when an oligonucleotide or polynucleotide is described as “comprising” a nucleoside or nucleotide described herein, it means that the nucleoside or nucleotide described herein forms a covalent bond with the oligonucleotide or polynucleotide. Similarly, when a nucleoside or nucleotide is described as part of an oligonucleotide or polynucleotide, such as “incorporated into” an oligonucleotide or polynucleotide, it means that the nucleoside or nucleotide described herein forms a covalent bond with the oligonucleotide or polynucleotide. In some such embodiments, the covalent bond is formed between a 3′ hydroxy group of the oligonucleotide or polynucleotide with the 5′ phosphate group of a nucleotide described herein as a phosphodiester bond between the 3′ carbon atom of the oligonucleotide or polynucleotide and the 5′ carbon atom of the nucleotide.

As used herein, the term “cleavable linker” is not meant to imply that the whole linker is required to be removed. The cleavage site can be located at a position on the linker that ensures that part of the linker remains attached to the detectable label and/or nucleoside or nucleotide moiety after cleavage.

As used herein, “derivative” or “analog” means a synthetic nucleotide or nucleoside derivative having modified base moieties and/or modified sugar moieties. Such derivatives and analogs are discussed in, e.g., Scheit, Nucleotide Analogs (John Wiley & Son, 1980) and Uhlman et al., Chemical Reviews 90:543-584, 1990. Nucleotide analogs can also comprise modified phosphodiester linkages, including phosphorothioate, phosphorodithioate, alkyl-phosphonate, and phosphoramidate linkages. “Derivative,” “analog” and “modified” as used herein, may be used interchangeably, and are encompassed by the terms “nucleotide” and “nucleoside” defined herein.

As used herein, the term “phosphate” is used in its ordinary sense as understood by those skilled in the art, and includes its protonated forms (for example,

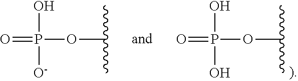 [SMILES: *OP(=O)([O-])O ; *OP(=O)(O)O]

As used herein, the terms “monophosphate,” “diphosphate,” and “triphosphate” are used in their ordinary sense as understood by those skilled in the art and include protonated forms.

As understood by one of ordinary skill in the art, a compound such as a nucleotide conjugate described herein may exist in ionized form, e.g., containing a —CO₂⁻, —SO₃⁻ or —O⁻. If a compound contains a positively or negatively charged substituent group, it may also contain a negatively or positively charged counterion such that the compound as a whole is neutral. In other aspects, the compound may exist in a salt form, where the counterion is provided by a conjugate acid or base.

As used herein, the term “phasing” refers to a phenomenon in SBS that is caused by incomplete removal of the 3′ terminators and fluorophores, and/or failure to complete the incorporation of a portion of DNA strands within clusters by polymerases at a given sequencing cycle. Prephasing is caused by the incorporation of nucleotides without effective 3′ terminators, wherein the incorporation event goes 1 cycle ahead due to a termination failure. Phasing and prephasing cause the measured signal intensities for a specific cycle to consist of the signal from the current cycle as well as noise from the preceding and following cycles. As the number of cycles increases, the fraction of sequences per cluster affected by phasing and prephasing increases, hampering the identification of the correct base. Prephasing can be caused by the presence of a trace amount of unprotected or unblocked 3′-OH nucleotides during sequencing by synthesis (SBS). The unprotected 3′-OH nucleotides could be generated during the manufacturing processes or possibly during the storage and reagent handling processes.

Methods of Sequencing with Post Incorporation Labeling

Certain embodiments of the present disclosure relates to a method of determining the sequences of a plurality of different target polynucleotides, comprising:
(a) contacting a solid support with a solution comprising sequencing primers under hybridization conditions, wherein the solid support comprises a plurality of different target polynucleotides immobilized thereon; and the sequencing primers are complementary to at least a portion of the target polynucleotides;
(b) contacting the solid support with an incorporation mixture comprising DNA polymerase and one more of four different types of nucleotides (A, C, G, and T or U; dATP, dCTP, dGTP and dTTP or dUTP) under conditions suitable for DNA polymerase-mediated primer extension, and incorporating one type of nucleotides into the sequencing primers to produce extended copy polynucleotides; wherein each of the four types of nucleotides comprises a 3′ blocking group; and the incorporation mixture comprises a first type of unlabeled nucleotide having a first functional moiety covalently attached to the first type of unlabeled nucleotide;
(c) contacting the extended copy polynucleotides with a first labeling reagent comprising a first detectable label and a first reactive moiety that reacts specifically with the first functional moiety of the first type of unlabeled nucleotides to form covalent bonding;
(d) imaging the solid support and performing one or more fluorescent measurements of the extended copy polynucleotides; and
(e) removing the 3′ blocking group of the incorporated nucleotides.

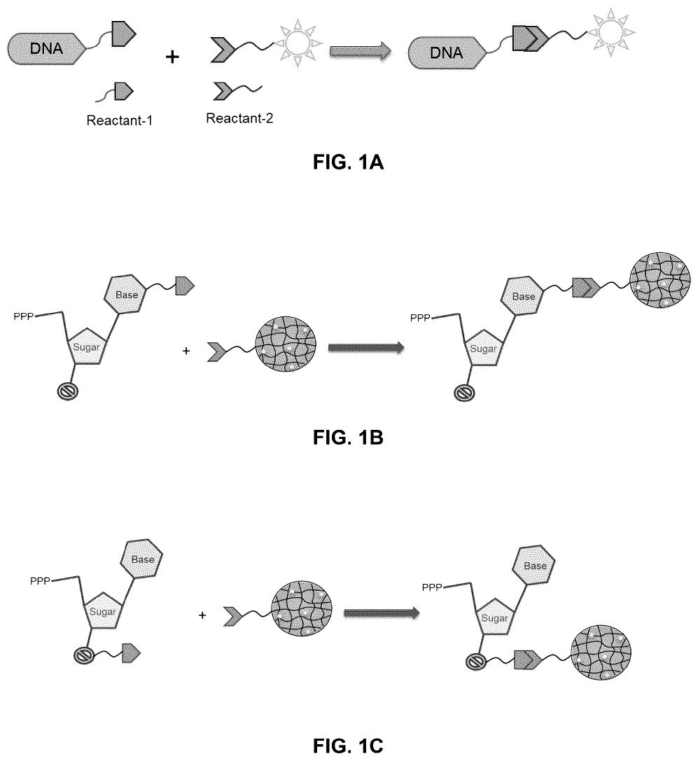

FIG. 1A illustrates a general scheme of bio-orthogonal post incorporation labeling method in sequencing by synthesis (SBS) described herein. In FIG. 1A, an unlabeled nucleotide is first incorporated into an extended copy polynucleotide (e.g., DNA) and the unlabeled nucleotide contains a functional moiety covalently attached thereto (i.e., Reactant-1). Then a labeling reagent (a detectable label containing a reactive moiety (i.e., Reactant-2)) is introduced to the DNA to label the incorporated unlabeled nucleotide through a chemical reaction between Reactant-1 and Reactant-2.

In some embodiments of the method described herein, the first functional moiety is covalently attached to the nucleobase of the first type of unlabeled nucleotide via a cleavable linker. In some other embodiments, the first functional moiety is covalently attached to the 3′ blocking group of the first type of unlabeled nucleotide via a cleavable linker.

FIG. 1B illustrates an example of the post incorporation labeling method described in FIG. 1A in which the detectable label is covalently attached to the nucleobase of the incorporated nucleotide. FIG. 1C illustrates another example of bio-orthogonal post incorporation labeling method described in FIG. 1A in which the detectable label is covalently attached to the 3′ position of the 2′-deoxyribose of the incorporated nucleotide.

In some embodiments of the method described herein, the first reactive moiety of the first labeling reagent forms covalent bonding with the first functional moiety of the first type of unlabeled nucleotides via a biorthogonal reaction selected from the group consisting of a [3+2] dipolar cycloaddition, a Diels-Alder cycloaddition, a [4+1] cycloaddition, a phosphine ligation, or condensation with 2-acylphenyl boronic acid. For example, a Diels-Alder cycloaddition may include inverse electron demand Diels-Alder cycloadditions (iEDDA). Various biorthogonal chemical reactions have been reported by Smeenk et al., Current Opinion in Chemical Biology 2021, 60:79-88, which is incorporated by reference in its entirety). The general reaction schemes for these reactions are illustrated as follows:

### 1. [3+2] Dipolar Cycloaddition

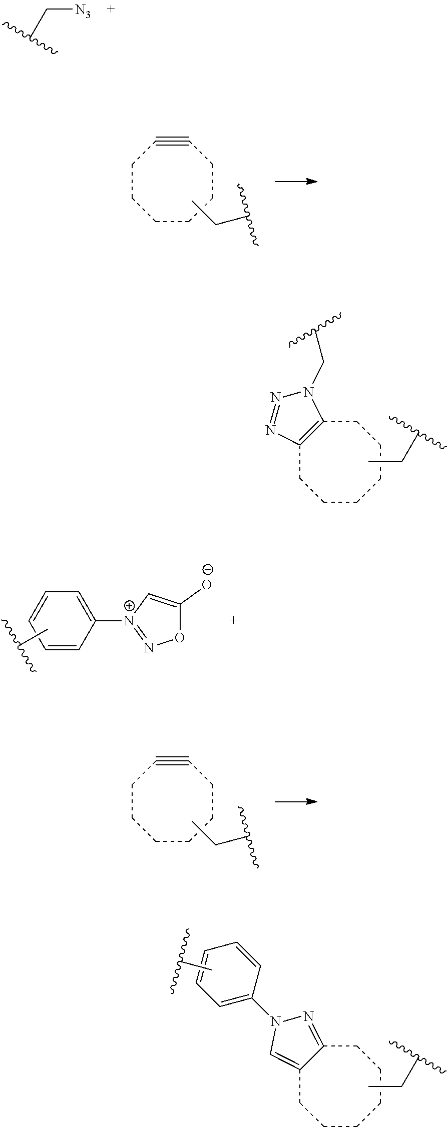

### 2. Inverse Electron Demand Diels-Alder Cycloaddition

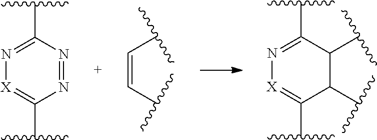

3. [4+1] Cycloaddition with Isonitrile

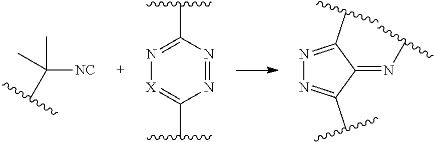

### 4. Phosphine Ligation

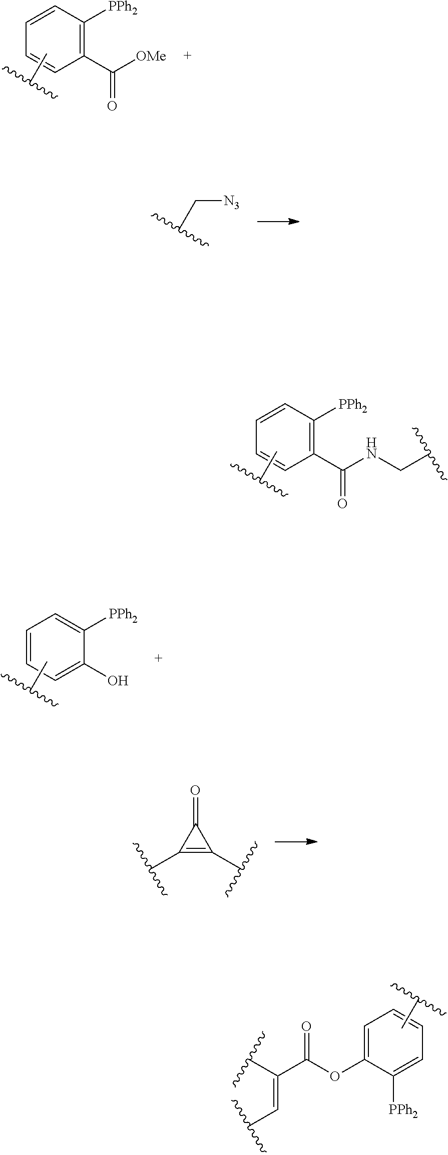

### 5. 2-Acylphenylboronic Acid Condensation

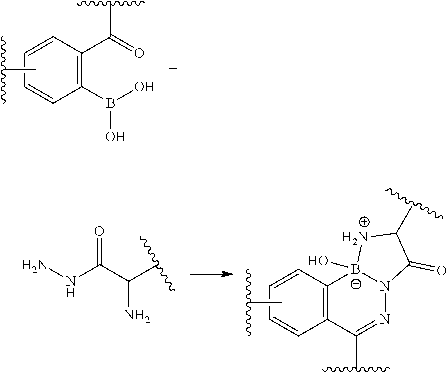

In some further embodiments, one of the first functional moiety and the first reactive moiety of the first labeling reagent comprises or is selected from azido, amino, unsubstituted or substituted vinyl, unsubstituted or substituted cyclopenta-1,3-dienyl, —S(O)₂CH—CH₂, —O—(CH₂)₂—SCH═CH₂, unsubstituted or substituted tetrazine, sydnone, imino sydnone, nitrone, unsubstituted or substituted triazine, cyclopropenone, cyclopropenium ion, 1,3-dithiolium-4-olate (DTO), chloro-oxime, amino hydrazine, or

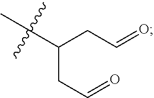 [SMILES: *C(CC=O)CC=O]

and the other one of the first functional moiety and the first reactive moiety of the first labeling reagent comprises or is selected from —O—NH₂, —SH, —NH—NH₂, unsubstituted or substituted alkynyl, unsubstituted or substituted C₅-C₁₆ cycloalkynyl, unsubstituted or substituted 5 to 16 membered heterocycloalkynyl, unsubstituted or substituted C₅-C₁₆ cycloalkenyl, unsubstituted or substituted 5 to 16 membered heterocycloalkenyl, substituted vinyl, isonitrile, substituted boronic acid moiety, substituted phosphines,

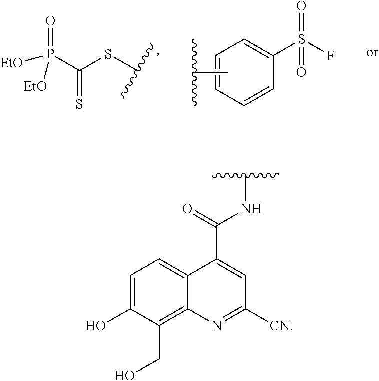

In particular, C₅-C₁₆ cycloalkynyl, 5 to 16 membered heterocycloalkynyl, C₅-C₁₆ cycloalkenyl, or 5 to 16 membered heterocycloalkenyl may undergo strain-promoted [3+2] cycloaddition reaction. Non-limiting examples include dibenzocyclooctyne (DBCO) having the structure

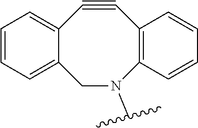 [SMILES: *N1Cc2ccccc2C#Cc2ccccc21]

such as

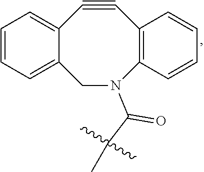 [SMILES: *C(=O)N1Cc2ccccc2C#Cc2ccccc21]

bicyclo[6.1.0] nonyne (BCN), norbornene, and transcyclooctene (TCO).

In some embodiments, one of the first functional moiety and the first reactive moiety comprises or is selected from alkynyl, unsubstituted or substituted DBCO moiety, unsubstituted or substituted BCN moiety, unsubstituted or substituted norbornene moiety, unsubstituted or substituted TCO moiety, primary isonitrile

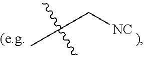

tertiary isonitrile

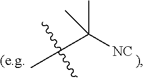

vinyl boronic acid

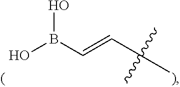 [SMILES: */C=C/B(O)O]

2-acylphenyl boronic acid

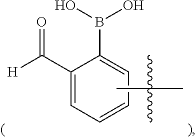

phosphine methyl ester

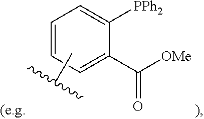

phosphine alcohol

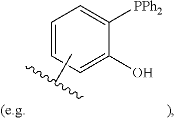

phosphine amine

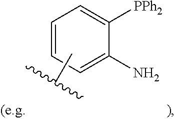

or phosphine thiol

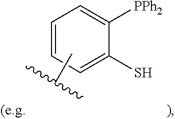

and the other one of the first functional moiety and the first reactive moiety comprises or is selected from azido (e.g., primary azide such as

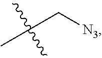 [SMILES: *CN=[N+]=[N-]]

aryl azide such as

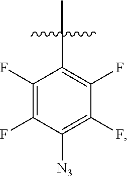 [SMILES: *c1c(F)c(F)c(N=[N+]=[N-])c(F)c1F]

tertiary azide such as

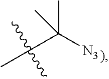 [SMILES: *C(C)(C)N=[N+]=[N-]]

sydnone

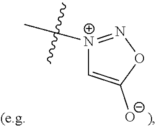 [SMILES: *[n+]1cc([O-])on1]

imino
sydnone

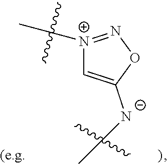 [SMILES: *[N-]c1c[n+](*)no1]

nitrone, phenyl tetrazine

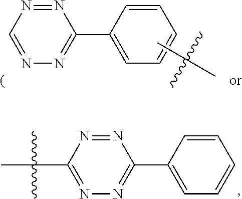

wherein the phenyl ring may be optionally substituted), pyrimidyl tetrazine

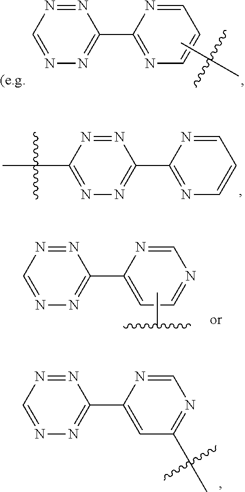

wherein the pyrimidyl ring may be optionally substituted), methyl tetrazine

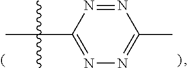

pyridyl tetrazine

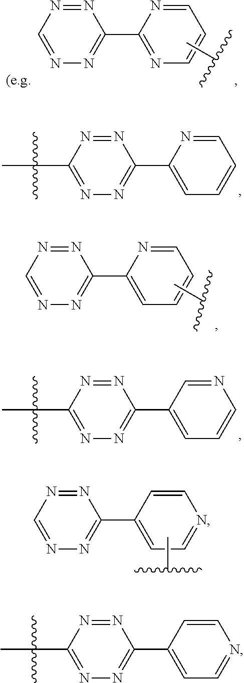

where the pyridyl ring may be optionally substituted), t-butyl tetrazine

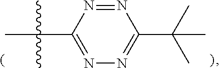

triazine, cyclopropenone

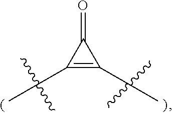

cyclopropenium ion

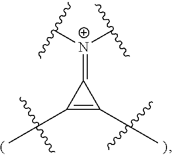

### DTO

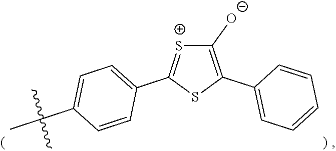

chloro-oxime

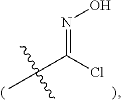

or amino hydrazine

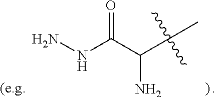 [SMILES: *C(N)C(=O)NN]

In some further embodiments, one of the first functional moiety and the first reactive moiety comprises or is selected from norbornene, TCO, DBCO, BCN, or optionally substituted triphenylphosphine, and the other one of the first functional moiety and the first reactive moiety comprises or is azido. For example, the first functional moiety comprises or is norbornene, TCO, DBCO, or BCN, and the first reactive moiety comprises or is azido. In other embodiments, the first functional moiety comprises or is azido, and the first reactive moiety comprises or is selected from norbornene, TCO, DBCO, or BCN moiety. In some other embodiments, one of the first functional moiety and the first reactive moiety comprises or is isonitrile, and the other one of the first functional moiety and the first reactive moiety comprises or is a substituted tetrazine. In some other embodiments, one of the first functional moiety and the first reactive moiety comprises or is TCO, and the other one of the first functional moiety and the first reactive moiety comprises or is a substituted tetrazine described herein. In some other embodiments, one of the first functional moiety and the first reactive moiety comprises or is an amino hydrazine moiety, and the other one of the first functional moiety and the first reactive moiety comprises or is a 2-acylphenyl boronic acid moiety.

In some embodiments of the method described herein, the incorporation mixture comprises a second type of labeled nucleotide, and a third type of labeled nucleotide.

In some other embodiments of the method described herein, the incorporation mixture comprises a second type of unlabeled nucleotide having a second functional moiety covalently attached to the second type of unlabeled nucleotide, and a third type of labeled nucleotide. In some other embodiments, the incorporation mixture comprises a second type of unlabeled nucleotide having a second functional moiety covalently attached to the second type of unlabeled nucleotide, and a mixture of a third type of unlabeled nucleotide having a first functional moiety covalently attached to the third type of unlabeled nucleotide and a third type of unlabeled nucleotide having a second functional moiety covalently attached to the third type of unlabeled nucleotide. In some such embodiments, step (c) further comprises contacting the extended copy polynucleotides with a second labeling reagent comprising a second detectable label and a second reactive moiety that reacts specifically with the second functional moiety to form covalent bonding. The second functional moiety and the second reactive moiety can undergo a biorthogonal reaction as described above with respect to the reaction between the first functional moiety and the first reactive moiety. For example, the first functional moiety/first reactive moiety pair can be one of unsubstituted or substituted norbornene, DBCO, or BCN/azido, and the second functional moiety/second reactive moiety pair can be unsubstituted or substituted TCO/unsubstituted or substituted tetrazine. In further embodiments, the third type of unlabeled nucleotide may carry both the first and the second functional moieties (e.g., DBCO and TCO moieties).

In some other embodiments of the method described herein, the incorporation mixture comprises a second type of unlabeled nucleotide having a second functional moiety covalently attached to the second type of unlabeled nucleotide, and a third type of unlabeled nucleotide having a third functional moiety covalently attached to the third type of unlabeled nucleotide. In some such embodiments, step (c) further comprises contacting the extended copy polynucleotides with a second labeling reagent comprising a second detectable label and a second reactive moiety that reacts specifically with the second functional moiety of the second type of unlabeled nucleotides to form covalent bonding, and a third labeling reagent comprising a third detectable label and a third reactive moiety that reacts specifically with the third functional moiety of the third type of unlabeled nucleotides to form covalent bonding. The third functional moiety and the third reactive moiety can undergo a biorthogonal reaction as described above with respect to the reaction between the first functional moiety and the first reactive moiety.

In some embodiments of the method described herein, the incorporation mixture comprises a fourth type of unlabeled nucleotide, wherein the fourth type of unlabeled nucleotide is not capable of reacting with any of the labeling reagent (e.g., the first, the second or the third labeling reagent).

In some embodiments of the method described herein, step (e) also removes the detectable labels of the incorporated nucleotides. In some such embodiments, the detectable labels and the 3′ blocking groups of the incorporated nucleotides are removed in a single chemical reaction. In some embodiments, the method further comprises step (f) washing the solid support with an aqueous wash solution. In some embodiments, steps (b) to (f) are repeated at least 50, 100, 150, 200, 250, 300, 350, 400, 450, or 500 cycles to determine the target polynucleotide sequences. In some further embodiments, the four types of nucleotides comprise dATP, dCTP, dGTP and dTTP or dUTP, or non-natural nucleotide analogs thereof.

In some embodiments of the method described herein, the first labeling reagent is contacted with the extended copy polynucleotides in step (c) by flushing the first labeling reagent through the extended copy polynucleotides. For example, the first labeling reagent is flushed through the extended copy polynucleotides in less than 5 s, 4.5 s, 4 s, 3.5 s, 3 s, 2.5 s, 2 s, 1.5 s or 1 s. In some further embodiments, the flush through of the first labeling reagent is conducted at an elevated temperature (e.g., at 40, 50 or 60° C.). In some further embodiments, when the method uses both a first labeling reagent and a second labeling reagent as described herein, the first and the second labeling reagents may be flushed through the extended copy polynucleotides, optionally at an elevated temperature as described herein simultaneously or sequentially. In some further embodiments, when the method uses a first labeling reagent, a second labeling reagent and a third labeling reagent as described herein, the first, the second, and the third labeling reagents may be flushed through the extended copy polynucleotides, optionally at an elevated temperature as described herein simultaneously or sequentially. In other embodiments, the labeling reagent(s) may be incubated with the extended copy polynucleotides for an extended period of time (e.g., 10 s, 15 s, 20 s, 25 s or 30 s). In some embodiments, one or more of the first labeling reagent, the second labeling reagent and the third labeling reagent are in an aqueous post incorporation labeling mixture. The mixture may contain additional inorganic salt(s) and buffering agents, including but not limited to NaCl, KCl, and citrate. The pH of the post incorporation labeling mixture may have a pH from about 7.0 to about 8.5, or from about 7.2 to about 8.0, or about 7.5.

In some embodiments of the method described herein, after step (b) the extended copy polynucleotides on the solid support are washed with a high salt buffer solution prior to contacting with the post incorporation labeling mixture. It is known that polymerase may stick to the polynucleotide strands after incorporation. Using a wash buffer with high concentration of salt is beneficial to the polymerase removal. Without being bound by a particular theory, it is believed that post incorporation labeling reaction kinetic can be improved by removing steric hinderance caused by polymerase sticking on the polynucleotide strands. In some embodiments, the high salt buffer solution contains a total concentration of salt or salts of at least 0.5M, 0.6M, 0.7M, 0.8M, 0.9M, 1M, 1.2M, 1.4M, 1.6M, 1.8M or 2M. The salt may be an inorganic alkaline metal salt including but not limited to NaCl, KCl, etc. In some further embodiments, after step (c) the labeled extended copy polynucleotides on the solid support are washed with a buffer solution to remove any remaining excess and/or unreacted labeling reagent(s), prior to imaging the solid support. In some such embodiments, the high salt buffer solution used before step (c) may also be used after the post incorporation labeling step. In some further embodiments, the high salt buffer solution may also be used as a wash buffer or wash solution in step (f). In some embodiments, the high salt buffer used after the incorporation step and the wash buffer/wash solution used in step (f) may contain additional components such as one or more Pd scavengers described herein.

In some embodiments of the method described herein, the method is performed on an automated sequencing instrument comprising two light sources operating at two different wavelengths. In some such embodiments, one light source has a wavelength of about 450 nm to about 460 nm, and the other light source has a wavelength of about 520 nm to about 540 nm. In other embodiments, the method is performed on an automated sequencing instrument having a single light source. In some such embodiments, the light source has a wavelength of about 450 nm to about 460 nm, or a wavelength of about 520 nm to about 540 nm.

In some embodiments of the method described herein, the incorporation of the nucleotide conjugates is performed by a mutant of 9°N polymerase, such as an archaeal family B polymerase, including but not limited to those disclosed in WO 2005/024010, U.S. Publication Nos. 2020/0131484 A1, 2020/0181587 A1, and 2024/0141427, each of which is incorporated by reference in its entirety. Exemplary polymerases include but not limited to Pol 812, Pol 1901, Pol 1558, Pol 963, or Pol A. The amino acid sequences of Pol 812, Pol 1901, Pol 1558 or Pol 963 DNA polymerases are described, for example, in U.S. Patent Publication Nos. 2020/0131484 A1 and 2020/0181587 A1. The amino acid sequence of Pol A is disclosed as SEQ ID NO:5 of U.S. Publication No. 2024/0141427.

In any embodiments of the method described herein, the density of the immobilized different target polynucleotides (i.e., clusters) on the solid support is about or at least about 50,000/mm², 100,000/mm², 150,000/mm², 200,000/mm², 250,000/mm², 300,000/mm², 350,000/mm², or 400,000/mm².

### Kits

Some embodiments of the present disclosure relate to a kit for sequencing application, comprising:

an incorporation mixture composition comprising one or more of four different types of nucleotides each comprising a 3′ blocking group, wherein a first type of unlabeled nucleotide having a first functional moiety covalently attached to the first type of unlabeled nucleotide; and a first labeling reagent comprising a first detectable label and a first reactive moiety that is capable of reacting specifically with the first functional moiety to form covalent bonding.

In some embodiments of the kit described herein, the first functional moiety is covalently attached to the nucleobase of the first type of unlabeled nucleotide via a cleavable linker. In other embodiments, the first functional moiety is covalently attached to the 3′ blocking group of the first type of unlabeled nucleotide via a cleavable linker.

In some embodiments of the kits described herein, the first reactive moiety of the first labeling reagent is capable of forming covalent bonding with the first functional moiety of the first type of unlabeled nucleotides via a biorthogonal reaction selected from the group consisting of a [3+2] dipolar cycloaddition, a Diels-Alder cycloaddition, a [4+1] cycloaddition, a phosphine ligation, or condensation with 2-acylphenyl boronic acid. The general reaction schemes of these reactions are described herein. In some embodiments, one of the first functional moiety and the first reactive moiety of the first labeling reagent comprises or is selected from azido, amino, unsubstituted or substituted vinyl, unsubstituted or substituted cyclopenta-1,3-dienyl, —S(O)₂CH═CH₂, —O—(CH₂)₂—SCH═CH₂, unsubstituted or substituted tetrazine, sydnone, imino sydnone, nitrone, unsubstituted or substituted triazine, cyclopropenone, cyclopropenium ion, 1,3-dithiolium-4-olate (DTO), chloro-oxime, amino hydrazine, or

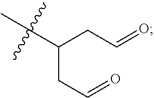 [SMILES: *C(CC=O)CC=O]

and the other one of the first functional moiety and the first reactive moiety of the first labeling reagent comprises or is selected from —O—NH₂, —SH, —NH—NH₂, unsubstituted or substituted alkynyl, unsubstituted or substituted C₅-C₁₆ cycloalkynyl, unsubstituted or substituted 5 to 16 membered heterocycloalkynyl, unsubstituted or substituted C₅-C₁₆ cycloalkenyl, unsubstituted or substituted 5 to 16 membered heterocycloalkenyl, substituted vinyl, isonitrile, substituted boronic acid moiety, substituted phosphines,

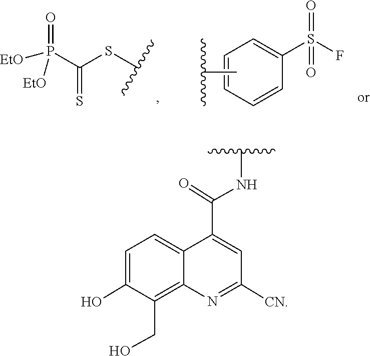

In some further embodiments, one of the first functional moiety and the first reactive moiety comprises or is selected from alkynyl, unsubstituted or substituted DBCO moiety, unsubstituted or substituted BCN moiety, unsubstituted or substituted norbornene moiety, unsubstituted or substituted TCO moiety, primary isonitrile, tertiary isonitrile, vinyl boronic acid, 2-acylphenyl boronic acid, phosphine methyl ester, phosphine alcohol, phosphine amine, or phosphine thiol, and the other one of the first functional moiety and the first reactive moiety comprises or is selected from azido, sydnone, imino sydnone, nitrone, pyrimidyl tetrazine, methyl tetrazine, pyridyl tetrazine, t-butyl tetrazine, optionally substituted phenyl tetrazine, triazine, cyclopropenone, cyclopropenium ion, DTO, chloro-oxime, or amino hydrazine. In some further embodiments, one of the first functional moiety and the first reactive moiety comprises or is selected from norbornene, TCO, DBCO, BCN, or optionally substituted triphenylphosphine moiety described herein (e.g., phosphine methyl ester, phosphine alcohol, phosphine amine, or phosphine thiol), and the other one of the first functional moiety and the first reactive moiety comprises or is azido. In some further embodiments, the first functional moiety comprises or is selected from norbornene, TCO, DBCO, or BCN moiety, and the first reactive moiety of the first labeling reagent comprises or is azido. In some other embodiments, the first functional moiety comprises or is azido, and the first reactive moiety of the first labeling reagent comprises or is selected from norbornene, TCO, DBCO, or BCN moiety. In some other embodiments, one of the first functional moiety and the first reactive moiety comprises or is isonitrile, and the other one of the first functional moiety and the first reactive moiety comprises or is a substituted tetrazine described herein (e.g., pyrimidyl tetrazine, methyl tetrazine, pyridyl tetrazine, t-butyl tetrazine, or optionally substituted phenyl tetrazine) In some further embodiments, one of the first functional moiety and the first reactive moiety comprises or is TCO, and the other one of the first functional moiety and the first reactive moiety comprises or is a substituted tetrazine described herein. In still some other embodiments, one of the first functional moiety and the first reactive moiety comprises or is an amino hydrazine moiety, and the other one of the first functional moiety and the first reactive moiety comprises or is a 2-acylphenyl boronic acid moiety described herein.

In some further embodiments, the kit comprises:
the first type of unlabeled nucleotide having the first functional moiety covalently attached to the first type of unlabeled nucleotide as described herein;
a second type of nucleotide;
a third type of nucleotide;
a fourth type of unlabeled nucleotide; and
the first labeling reagent comprising a reactive moiety that is capable of reacting specifically with the first functional moiety to form covalent bonding;
wherein the fourth type of unlabeled nucleotide is not capable of reacting with the first labeling reagent.

In some such embodiments of the kit described herein, each of the second type of nucleotide and the third type of nucleotide is labeled. In some other embodiments, the second type of nucleotide is a second type of unlabeled nucleotide having a second functional moiety covalently attached to the second type of unlabeled nucleotide, and the kit further comprises a second labeling reagent comprising a second detectable label and a second reactive moiety that is capable of reacting specifically with the second functional moiety to form covalent bonding, and wherein the fourth type of unlabeled nucleotide is not capable of reacting with either the first labeling reagent or the second labeling reagent. In some such embodiments, the third type of nucleotide is labeled. In some other such embodiments, the third type of nucleotide is a mixture of a third type of unlabeled nucleotide having a first functional moiety covalently attached to the third type of unlabeled nucleotide and a third type of unlabeled nucleotide having a second functional moiety covalently attached to the third type of unlabeled nucleotide. For example, the first functional moiety/first reactive moiety pair can be one of unsubstituted or substituted norbornene, DBCO, or BCN/azido, and the second functional moiety/second reactive moiety pair can be unsubstituted or substituted TCO/unsubstituted or substituted tetrazine. In further embodiments, the third type of unlabeled nucleotide may carry both the first and the second functional moieties (e.g., DBCO and TCO moieties). In some other such embodiments, the third type of nucleotide is a third type of unlabeled nucleotide having a third functional moiety covalently attached to the third type of unlabeled nucleotide, and the kit further comprises a third labeling reagent comprising a third detectable label and a third reactive moiety that is capable of reacting specifically to the third functional moiety of the third type of unlabeled nucleotide to form covalent bonding, and wherein the fourth type of unlabeled nucleotide is not capable of reacting with any one of the first labeling reagent, the second labeling reagent, or the third labeling reagent. The second functional moiety and the second reactive moiety can undergo a biorthogonal reaction as described above with respect to the reaction between the first functional moiety and the first reactive moiety. The third functional moiety and the third reactive moiety can undergo a biorthogonal reaction as described above with respect to the reaction between the first functional moiety and the first reactive moiety. For example, the kit may comprise:
a first type of unlabeled nucleotide having a first functional moiety covalently attached to the first type of unlabeled nucleotide;
a second type of unlabeled nucleotide having a second functional moiety covalently attached to the second type of unlabeled nucleotide;
a mixture of a third type of unlabeled nucleotide having a first functional moiety covalently attached to the third type of unlabeled nucleotide and a third type of unlabeled nucleotide having a second functional moiety covalently attached to the third type of unlabeled nucleotide;
a fourth type of unlabeled nucleotide;
a first labeling reagent comprising a first reactive moiety that is capable of reacting specifically with the first functional moiety to form covalent bonding; and
a second labeling reagent comprising a second reactive moiety that is capable of reacting specifically with the second functional moiety to form covalent bonding;
   wherein the fourth type of unlabeled nucleotide is not capable of reacting with the first labeling reagent and the second labeling reagent. In some such embodiments the first functional moiety comprises or is DBCO and the first reactive moiety is azido, or vice versa. In some such embodiments, the second functional moiety comprises or is TCO and the second reactive moiety is a substituted tetrazine as described herein, or vice versa. In some other embodiments, the third type of nucleotide is a labeled nucleotide. In other embodiments, the third type of nucleotide is a mixture of a third type of unlabeled nucleotide having a DBCO moiety and a third type of unlabeled nucleotide having a TCO moiety. In some embodiments, one or more of the first labeling reagent, the second labeling reagent and the third labeling reagent are in an aqueous post incorporation labeling mixture. The mixture may contain additional inorganic salt(s) and buffering agents, including but not limited to NaCl, KCl, and citrate. The pH of the post incorporation labeling mixture may have a pH from about 7.0 to about 8.5, or from about 7.2 to about 8.0, or about 7.5.

In some embodiments, the incorporation mixture composition further comprises a DNA polymerase, such as a mutant of 9°N polymerase disclosed in WO 2005/024010, U.S. Publication Nos. 2020/0131484 A1, 2020/0181587 A1, and 2024/0141427, each of which is incorporated by reference in its entirety.

In some embodiments, the four different types of nucleotides are distinguishable using a single light source (e.g., a blue light source having a wavelength from about 450 nm to about 460 nm or a green light source having a wavelength from about 520 nm to about 540 nm). In some other embodiments, the four different types of nucleotides are distinguishable using two light sources operating at two different wavelengths. For example, one light source has a wavelength of about 450 nm to about 460 nm, and the other light source has a wavelength of about 520 nm to about 540 nm.

Additional embodiments of the present disclosure include a system for nucleic acid sequencing, comprising a plurality of chambers, wherein one of the chamber contains the kit described herein.

### Fluorescent Dyes

Various fluorescent dyes may be used in the present disclosure as detectable labels for the post incorporation labeling reagents described herein, in particularly those dyes that may be excitation by a blue light or a green light. These dyes may also be referred to as “blue dyes” and “green dyes” respectively. Examples of various type of blue dyes, including but not limited to coumarin dyes, chromenoquinoline dyes, naphthalimide dyes, and bisboron containing heterocycles are disclosed in U.S. Publication Nos. 2018/0094140, 2018/0201981, 2020/0277529, 2020/0277670, 2021/0188832 2022/0033900, 2022/0195517, 2022/0380389, 2023/0313292, 2023/0416279 and U.S. Ser. No. 18/618,509, each of which is incorporated by reference in its entirety. Non-limiting examples of the blue dyes include:

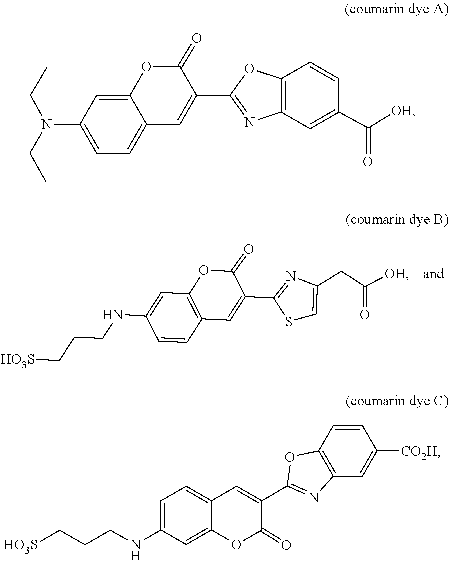 [SMILES: CCN(CC)c1ccc2cc(-c3nc4cc(C(=O)O)ccc4o3)c(=O)oc2c1 ; O=C(O)Cc1csc(-c2cc3ccc(NCCCS(=O)(=O)O)cc3oc2=O)n1 ; O=C(O)c1ccc2oc(-c3cc4ccc(NCCCS(=O)(=O)O)cc4oc3=O)nc2c1]

and salts, mesomeric forms, and optionally substituted analogs thereof. For example, analogs with —SO₃H substitution on the alkyl group(s).

Examples of green dyes including cyanine or polymethine dyes disclosed in International Publication Nos. WO2013/041117, WO2014/135221, WO2016/189287, WO2017/051201 and WO2018/060482A1, each of which is incorporated by reference in its entirety. Non-limiting examples of the green dyes include:

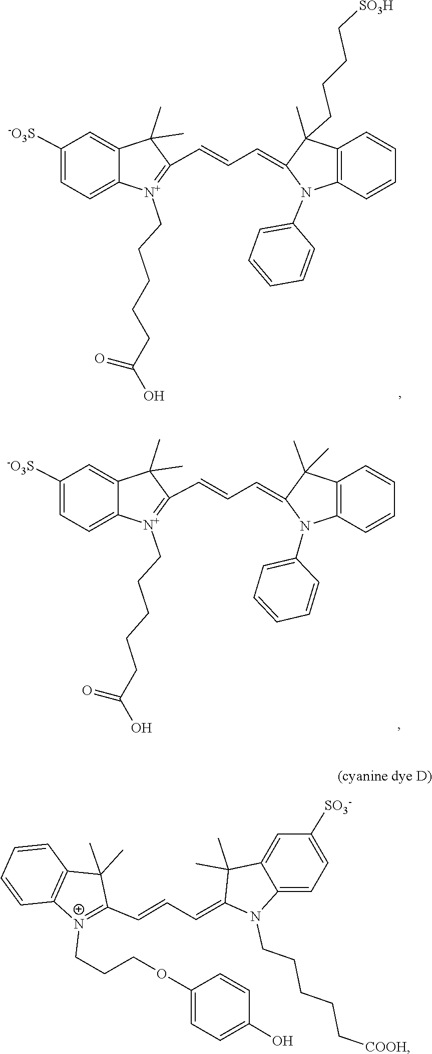

and salts, mesomeric forms, and optionally substituted analogs thereof.

In some embodiments, the fluorescent dyes described herein may be further modified to introduce one or more substituents (such as —SO₃H, —OH, —C(O)OH, —C(O)OR, where R is unsubstituted or substituted C₁-C₆ alkyl) to improve the hydrophilicity of the dyes while maintaining the signal intensity of the dye. In some such embodiments, coumarin dye A

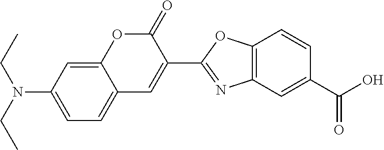 [SMILES: CCN(CC)c1ccc2cc(-c3nc4cc(C(=O)O)ccc4o3)c(=O)oc2c1]

may be further modified to improve the hydrophilicity of the compound as

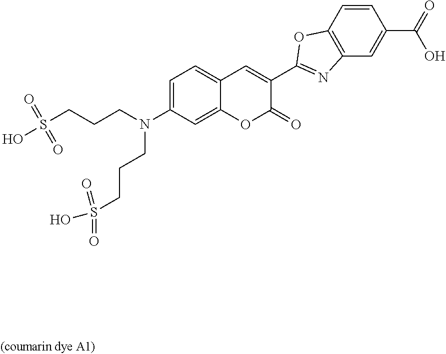 [SMILES: O=C(O)c1ccc2oc(-c3cc4ccc(N(CCCS(=O)(=O)O)CCCS(=O)(=O)O)cc4oc3=O)nc2c1]

or a salt thereof (where —SO₃H is in ionized form —SO₃).

### 3′ Blocking Groups

The nucleotide described herein may also have a 3′ blocking group covalently attached to the deoxyribose sugar of the nucleotide. Various 3′ blocking group are disclosed in WO2002/029003, WO2004/018497 and WO2014/139596. For example, the blocking group may be azidomethyl (—CH₂N₃) or substituted azidomethyl (e.g., —CH(CHF₂)N₃ or CH(CH₂F)N₃), or allyl, each connecting to the 3′-oxygen atom of the deoxyribose moiety. In some embodiments, the 3′ blocking group is azidomethyl, forming 3′-OCH₂N₃ with the 3′ carbon of the ribose or deoxyribose.

Additional 3′ blocking groups are disclosed in U.S. Publication No. 2020/0216891 A1, which is incorporated by reference in its entirety. Non-limiting examples of the acetal blocking group

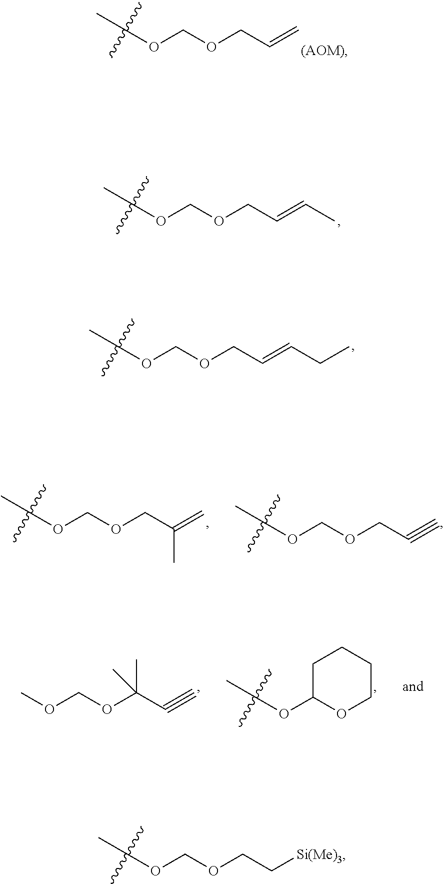

each covalently attached to the 3′ carbon of the deoxyribose.

### Deprotection of the 3′ Blocking Groups

In some embodiments, the 3′ hydroxy protecting group such as azidomethyl may be removed or deprotected by using a water-soluble phosphine reagent to generate a free 3′-OH. Non-limiting examples include tris(hydroxymethyl) phosphine (THMP), tris(hydroxyethyl) phosphine (THEP) or tris(hydroxypropyl) phosphine (THP or THPP). 3′-acetal blocking groups described herein may be removed or cleaved under various chemical conditions. For 3′ blocking groups that contain an allyl moiety, non-limiting cleaving condition includes a Pd(II) complex, such as Pd(OAc)₂ or allylPd(II) chloride dimer, in the presence of a phosphine ligand, for example tris(hydroxymethyl) phosphine (THMP), or tris(hydroxypropyl) phosphine (THP or THPP). For those blocking groups containing an alkynyl group (e.g., an ethynyl), they may also be removed by a Pd(II) complex (e.g., Pd(OAc) 2 or allyl Pd(II) chloride dimer) in the presence of a phosphine ligand (e.g., THP or THMP).

### Palladium Cleavage Reagents

In some other embodiments, the 3′ blocking group such as allyl or AOM as described herein may be cleaved by a palladium catalyst. In some such embodiments, the Pd catalyst is water soluble. In some such embodiments, is a Pd(0) complex (e.g., Tris(3,3′,3″-phosphinidynetris(benzenesulfonato) palladium (0) nonasodium salt nonahydrate). In some instances, the Pd(0) complex may be generated in situ from reduction of a Pd(II) complex by reagents such as alkenes, alcohols, amines, phosphines, or metal hydrides. Suitable palladium sources include Na₂PdCl₄, Li₂PdCl₄, Pd(CH₃CN)₂Cl₂. (PdCl(C₃H₅))₂, [Pd(C₃H₅)(THP)]Cl, [Pd(C₃H₅)(THP)₂]Cl, Pd(OAc)₂, Pd(Ph₃)₄, Pd(dba)₂, Pd(Acac)₂, PdCl₂ (COD), Pd(TFA) 2, Na₂PdBr₄, K₂PdBr₄, PdCl₂, PdBr₂, and Pd(NO₃)₂. In one such embodiment, the Pd(0) complex is generated in situ from Na₂PdCl₄ or K₂PdCl₄. In another embodiment, the palladium source is allyl palladium (II) chloride dimer [(PdCl(C₃H₅))₂]. In some embodiments, the Pd(0) complex is generated in an aqueous solution by mixing a Pd(II) complex with a phosphine. Suitable phosphines include water soluble phosphines, such as tris(hydroxypropyl)phosphine (THP), tris(hydroxymethyl) phosphine (THMP), 1,3,5-triaza-7-phosphaadamantane (PTA), bis(p-sulfonatophenyl)phenylphosphine dihydrate potassium salt, tris(carboxyethyl) phosphine (TCEP), and triphenylphosphine-3,3′,3″-trisulfonic acid trisodium salt.

In some embodiments, the palladium catalyst is prepared by mixing [(Allyl) PdCl]₂ with THP in situ. The molar ratio of [(Allyl) PdCl]₂ and the THP may be about 1:1, 1:1.5, 1:2, 1:2.5, 1:3, 1:3.5, 1:4, 1:4.5, 1:5, 1:5.5, 1:6, 1:6.5, 1:7, 1:7.5, 1:8, 1:8.5, 1:9, 1:9.5 or 1:10. In one embodiment, the molar ratio of [(Allyl) PdCl]₂ to THP is 1:10. In some other embodiment, the palladium catalyst is prepared by mixing a water soluble Pd reagent such as Na₂PdCl₄ or K₂PdCl₄ with THP in situ. The molar ratio of Na₂PdCl₄ or K₂PdCl₄ and THP may be about 1:1, 1:1.5, 1:2, 1:2.5, 1:3, 1:3.5, 1:4, 1:4.5, 1:5, 1:5.5, 1:6, 1:6.5, 1:7, 1:7.5, 1:8, 1:8.5, 1:9, 1:9.5 or 1:10. In one embodiment, the molar ratio of Na₂PdCl₄ or K₂PdCl₄ to THP is about 1:3. In another embodiment, the molar ratio of Na₂PdCl₄ or K₂PdCl₄ to THP is about 1:3.5. In yet another embodiment, the molar ratio of Na₂PdCl₄ or K₂PdCl₄ to THP is about 1:2.5. In some further embodiments, one or more reducing agents may be added, such as ascorbic acid or a salt thereof (e.g., sodium ascorbate). In some embodiments, the cleavage mixture may contain additional buffer reagents, such as a primary amine, a secondary amine, a tertiary amine, a carbonate salt, a phosphate salt, or a borate salt, or combinations thereof. In some further embodiments, the buffer reagent comprises ethanolamine (EA), tris(hydroxymethyl) aminomethane (Tris), glycine, sodium carbonate, sodium phosphate, sodium borate, 2-dimethylethanolamine (DMEA), 2-dicthylethanolamine (DEEA), N,N,N′,N′-tetramethylethylenediamine (TEMED), or N,N,N′,N′-tetraethylethylenediamine (TEEDA), or 2-piperidine ethanol (also known as (2-hydroxyethyl) piperidine, having the structure

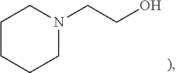

or combinations thereof. In one embodiment, the buffer reagent comprises or is DEEA. In another embodiment, the buffer reagent comprises or is (2-hydroxyethyl) piperidine. In another embodiment, the buffer reagent contains one or more inorganic salts such as a carbonate salt, a phosphate salt, or a borate salt, or combinations thereof. In one embodiment, the inorganic salt is a sodium salt.

### Palladium (Pd) Scavengers

Pd has the capacity to stick on DNA, mostly in its inactive Pd(II) form, which may interfere with the binding between DNA and polymerase, causing increased phasing. A post-cleavage wash composition that includes a Pd scavenger compound may be used following the deblocking step. For example, PCT Publication No. WO 2020/126593 discloses Pd scavengers such as 3,3′-dithiodipropionic acid (DDPA) and lipoic acid (LA) may be included in the scan composition and/or the post-cleavage wash composition. The use of these scavengers in the post-cleave washing solution has the purpose of scavenging Pd(0), converting Pd(0) to the inactive Pd(II) form, thereby improving the prephasing value and sequencing metrics, reducing signal degrade, and extend sequencing read length.

### Pd(0) Scavengers

Certain aspects of the present disclosure relate to employing alternative palladium scavengers in several steps of sequencing by synthesis, where at least one palladium scavenger comprises one or more allyl moieties. For example, —O-allyl, —S-allyl, —NR-allyl, or —N⁺RR′-allyl, or combinations thereof, wherein R is H, unsubstituted or substituted C₁-C₆ alkyl, unsubstituted or substituted C₂-C₆ alkenyl, unsubstituted or substituted C₂-C₆ alkynyl, unsubstituted or substituted C₆-C₁₀ aryl, unsubstituted or substituted 5 to 10 membered heteroaryl, unsubstituted or substituted C₃-C₁₀ carbocyclyl, or unsubstituted or substituted 5 to 10 membered heterocyclyl; and R′ is H, unsubstituted C₁-C₆ alkyl or substituted C₁-C₆ alkyl. The allyl containing Pd scavenger acts as a competitive substrate to consume any residual Pd(0) sticking on the nucleic acid (i.e., a Pd(0) scavenger). These palladium scavengers are described in WO 2022/243480, which is incorporated by reference in its entirety.

In some embodiments of any of the methods described herein, the Pd(0) scavenger comprises one or more allyl moieties is the incorporation mix (IMX). In some such embodiments, such palladium scavenger is compatible with the other sequencing reagents in the incorporation mix, which may also include a polymerase (such as DNA polymerase), in addition to the one or more different types of nucleotides. In some such embodiments, the polymerase is a DNA polymerase, such as a mutant of 9°N polymerase (e.g., those disclosed in WO 2005/024010, which is incorporated by reference), for example, Pol 812, Pol 1901, Pol 1558 or Pol 963. In some embodiments, the Pd(0) scavenger is premixed with the DNA polymerase and/or the one or more of four types of nucleotides (e.g., dATP, dCTP, dGTP, and dTTP or dUTP). In other embodiments, the Pd(0) scavenger is stored separately form the DNA polymerase and/or the one or more of four types of nucleotides and is mixed with these components shortly before sequencing run starts.

In some other embodiments of the methods and kits described herein, the Pd(0) scavenger comprises one or more allyl moieties is in a solution when performing one or more fluorescent measurements. In such embodiment, such palladium scavenger is compatible with the sequencing reagents of the scanning solution (also known as the scan mix). In further embodiments, the one or more palladium scavengers does not require a separate washing step prior to the next incorporation cycle. In further embodiments, the palladium scavenger in the scan solution is a Pd(0) scavenger described herein. In other embodiments of the methods described herein, the Pd(0) scavenger comprises one or more allyl moieties is in the post cleavage wash solution of step (f). In further embodiments, the palladium scavenger in the post cleavage wash solution is a Pd(0) scavenger described herein. In some such embodiment, the post cleavage wash solution does not comprise lipoic acid or 3,3′-dithiodipropionic acid (DDPA). In still other embodiments, the Pd(0) scavenger comprises one or more allyl moieties may be present both in the incorporation mix of step (b) and the post cleavage wash solution of step (f), or present in both the incorporation mix and the scan mix. In some such embodiment, the post cleavage wash solution comprises lipoic acid or DDPA. In other embodiments, the post cleavage wash solution does not comprise lipoic acid or DDPA.

Non-limiting examples of the Pd(0) scavenger comprising one or more —O-allyl or allyl moieties include the following:

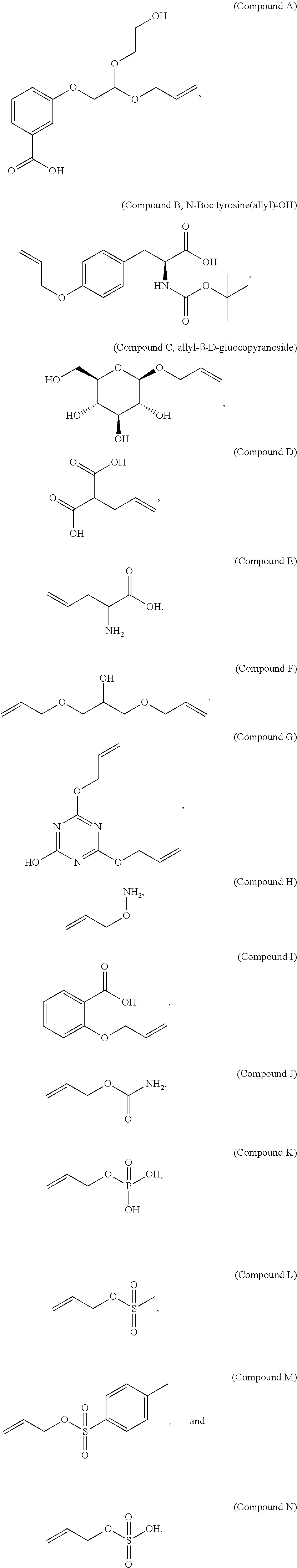

Non-limiting examples of the Pd(0) scavenger comprising one or more-S-allyl moieties include the following:

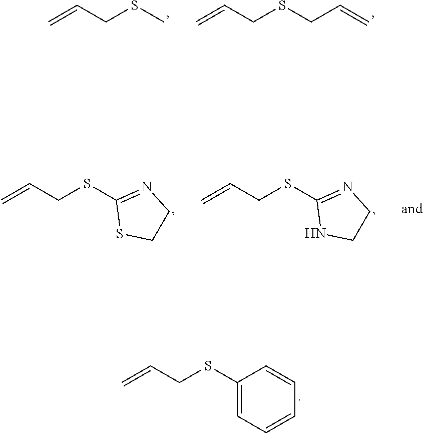

Non-limiting examples of the Pd(0) scavenger comprising one or more-NR-allyl or —N⁺RR′-allyl moieties include the following:

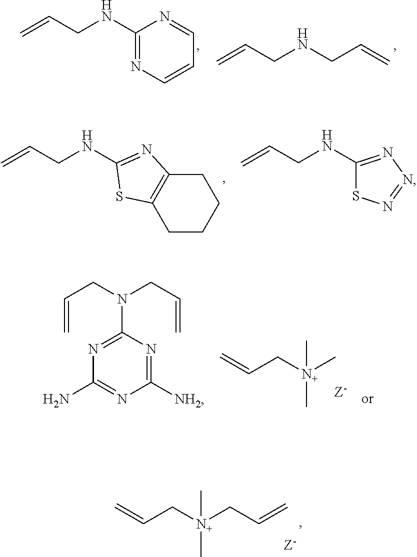

where Z⁻ is an anion (e.g., a halide anion such as F⁻ or Cl⁻). In one embodiment, the palladium scavenger is

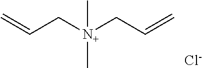

(Compound O, diallyldimethylammonium chloride, also known as DADMAC).

### Pd(II) Scavengers

In some embodiments of the methods and kits described herein, the method may further use additional palladium scavenger(s), such as Pd(II) scavenger(s). In some such embodiments, the use of additional Pd(II) scavenger(s) may improve the phasing value of the sequencing metrics. For example, the Pd(II) scavenger(s) may comprise an isocyanoacetate (ICNA) salt, ethyl isocyanoacetate, methyl isocyanoacetate, cysteine (e.g., L-cysteine) or a salt thereof (e.g., N-acetyl-L-cysteine), potassium ethylxanthogenate, potassium isopropyl xanthate, glutathione, ethylenediaminetetraacetic acid (EDTA), iminodiacetic acid, nitrilodiacetic acid, trimercapto-S-triazine, dimethyldithiocarbamate, dithiothreitol, mercaptoethanol, allyl alcohol, propargyl alcohol, thiol, thiosulfate salt (e.g., sodium thiosulfate or potassium thiosulfate), tertiary amine and/or tertiary phosphine, or combinations thereof. In one embodiment, the method also includes the use of L-cysteine or a salt thereof. In another embodiment, the method also includes the use of a thiosulfate salt such as sodium thiosulfate (Na₂S₂O₃). In some such embodiments, the Pd(II) scavenger (e.g., L-cysteine or sodium thiosulfate) is in the aqueous solution containing the DNA polymerase and the nucleotides (i.e., incorporation mix). In other embodiments, the Pd(II) scavenger (e.g., L-cysteine or sodium thiosulfate) is in the post cleavage wash solution. In other embodiments, the Pd(II) scavenger (e.g., L-cysteine or sodium thiosulfate) may be both present in the incorporation mixture. In other embodiments, the Pd(II) scavenger (e.g., L-cysteine or sodium thiosulfate) may be present in the scan mixture (i.e., the solution in which one or more fluorescent measurements of the incorporated nucleotide are performed). In other embodiments, the Pd(II) scavenger may be present in one or more of incorporation mixture (i.e., the aqueous solution of step (b)), the scan mixture, or the post-cleavage wash solution (i.e., the aqueous wash solution of step (f)).

### Cleavable Linkers

In some embodiments, the first/second/third functional moiety of nucleotide described herein is covalently attached to the nucleobase of the nucleotide via a cleavable linker. Use of the term “cleavable linker” is not meant to imply that the whole linker is required to be removed. The cleavage site can be located at a position on the linker that ensures that part of the linker remains attached to the dye and/or substrate moiety after cleavage. Cleavable linkers may be, by way of non-limiting example, electrophilically cleavable linkers, nucleophilically cleavable linkers, photocleavable linkers, cleavable under reductive conditions (for example disulfide or azide containing linkers), oxidative conditions, cleavable via use of safety-catch linkers and cleavable by elimination mechanisms. The use of a cleavable linker to attach the dye compound to a substrate moiety ensures that the label can, if required, be removed after detection, avoiding any interfering signal in downstream steps.

Useful linker groups may be found in PCT Publication No. WO2004/018493 (herein incorporated by reference), examples of which include linkers that may be cleaved using water-soluble phosphines or water-soluble transition metal catalysts formed from a transition metal and at least partially water-soluble ligands. In aqueous solution the latter form at least partially water-soluble transition metal complexes. Such cleavable linkers can be used to connect bases of nucleotides to labels such as the dyes set forth herein.

Particular linkers include those disclosed in PCT Publication No. WO2004/018493 (herein incorporated by reference) such as those that include moieties of the formulae:

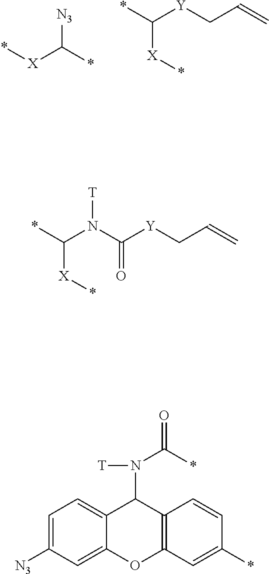

(wherein X is selected from the group comprising O, S, NH and NQ wherein Q is a C₁-10 substituted or unsubstituted alkyl group, Y is selected from the group comprising O, S, NH and N (allyl), T is hydrogen or a C₁-C₁₀ substituted or unsubstituted alkyl group and * indicates where the moiety is connected to the remainder of the nucleotide or nucleoside). In some aspect, the linkers connect the bases of nucleotides to labels such as, for example, the dye compounds described herein.

Additional examples of linkers include those disclosed in U.S. Publication No. 2016/0040225 (herein incorporated by reference), such as those include moieties of the formulae:

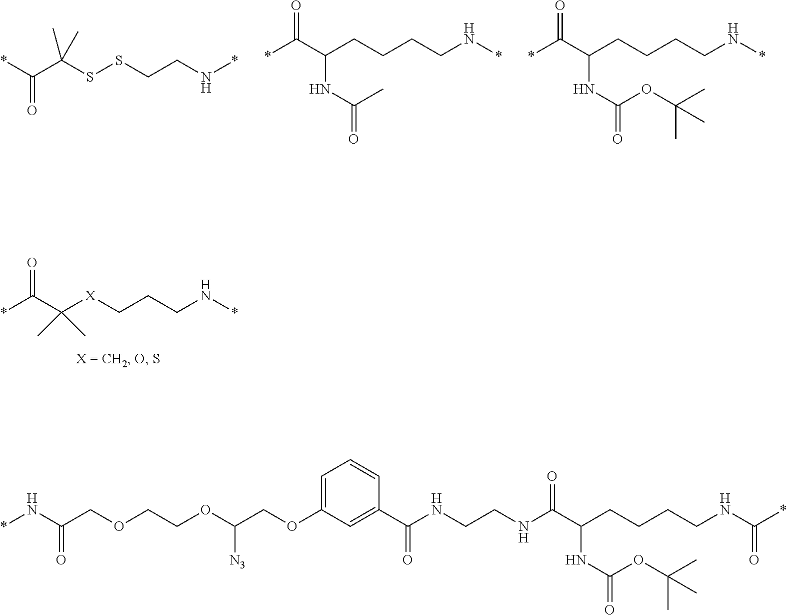

(wherein * indicates where the moiety is connected to the remainder of the nucleotide or nucleoside). The linker moieties illustrated herein may comprise the whole or partial linker structure between the nucleotides/nucleosides and the labels.

Additional examples of linkers are disclosed in U.S. Publication No. 2020/0216891 A1, which is incorporated by reference in its entirety:

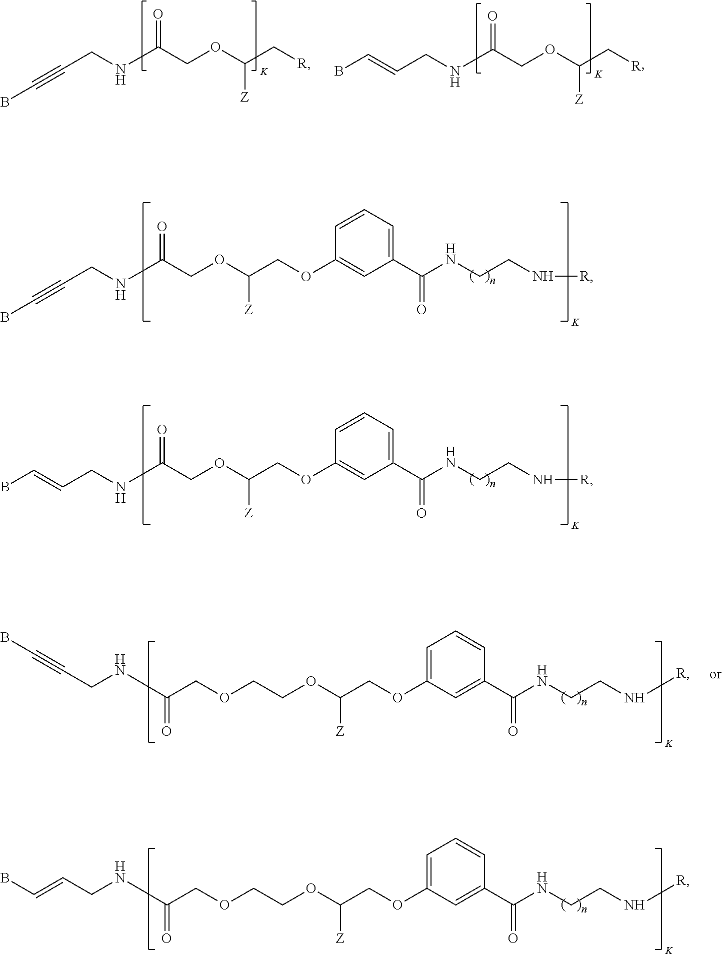

wherein B is a nucleobase; n is 1, 2, 3, 4, 5; k is 1; Z is —N₃ (azido), —O—C₁-C₆alkyl, O—C₂-C₆ alkenyl, or —O—C₂-C₆ alkynyl; and R comprises the first, the second, or the third functional moiety described herein, which may contain additional linker and/or spacer structure. One of ordinary skill in the art understands that the first, the second, or the third functional moiety described herein is covalently bound to the linker by reacting a functional group of the functional moiety containing compound (e.g., carboxyl) with a functional group of the linker (e.g., amino) to form an amide bond. In one embodiment, the cleavable linker comprises

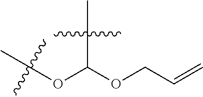 [SMILES: *OC(*)OCC=C]

(“AOL” linker moiety) where Z is-O-allyl. For the purpose of the present disclosure, the nucleotide may contain multiple cleavable linkers repeating units (e.g., k is 1, 2, 3, 45, 6, 7, 8, 9 or 10).

The first, second or third functional moiety may be attached to any position on the nucleotide base, for example, through a linker. In particular embodiments, Watson-Crick base pairing can still be carried out for the resulting analog. Particular nucleobase labeling sites include the C₅ position of a pyrimidine base or the C₇ position of a 7-deaza purine base. As described above a linker group may be used to covalently attach a dye to the nucleotide.

In particular embodiments, the unlabeled nucleotide may be enzymatically incorporable and enzymatically extendable. Accordingly, a linker moiety may be of sufficient length to connect the nucleotide to the compound such that the compound does not significantly interfere with the overall binding and recognition of the nucleotide by a nucleic acid replication enzyme. Thus, the linker can also comprise a spacer unit, such as one or more PEG unit(s) (—OCH₂CH₂—) n, where n is an integer of 1-20, for example 1, 2, 3, 4, 5, 6, 7, 8, 9, 10, 11, 12, 13, 14 or 15. The spacer distances, for example, the nucleotide base from a cleavage site or label.

A unlabeled nucleotides described herein may have the formula:

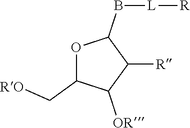

where R is the first, second or third functional moiety described herein; B is a nucleobase, such as, for example uracil, thymine, cytosine, adenine, 7-deaza adenine, guanine, 7-deaza guanine, and the like; L is a linker; —OR′ is monophosphate, diphosphate, triphosphate, thiophosphate, a phosphate ester analog, —O— attached to a reactive phosphorous containing group, or —O-protected by a blocking group; R″ is H or OH; and R″ is H, a 3′ hydroxy blocking group described herein, or —OR′″ forms a phosphoramidite. Where —OR″ is phosphoramidite, R′ is an acid-cleavable hydroxy protecting group which allows subsequent monomer coupling under automated synthesis conditions. In some further embodiments, B comprises

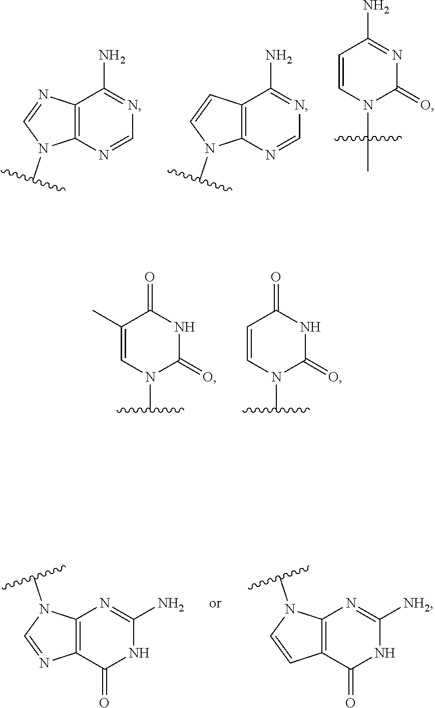

or optionally substituted derivatives and analogs thereof. In some further embodiments, the nucleobase comprises the structure

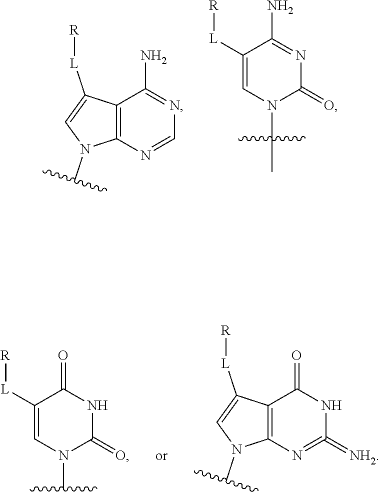

In yet another alternative embodiment, there is no blocking group on the 3′ carbon of the pentose sugar and the labeled avidin attached to the base via a linker, for example, can be of a size or structure sufficient to act as a block to the incorporation of a further nucleotide. Thus, the block can be due to steric hindrance or can be due to a combination of size, charge and structure, whether or not the dye is attached to the 3′ position of the sugar.

The use of a blocking group allows polymerization to be controlled, such as by stopping extension when an unlabeled nucleotide is incorporated. If the blocking effect is reversible, for example, by way of non-limiting example by changing chemical conditions or by removal of a chemical block, extension can be stopped at certain points and then allowed to continue.

In a particular embodiment, the linker and blocking group are both present and are separate moieties. In particular embodiments, the linker and blocking group are both cleavable under the same or substantially similar conditions. Thus, deprotection and deblocking processes may be more efficient because only a single treatment will be required to remove both the dye compound and the blocking group. However, in some embodiments a linker and blocking group need not be cleavable under similar conditions, instead being individually cleavable under distinct conditions.

Non-limiting exemplary unlabeled nucleotides as described herein include:

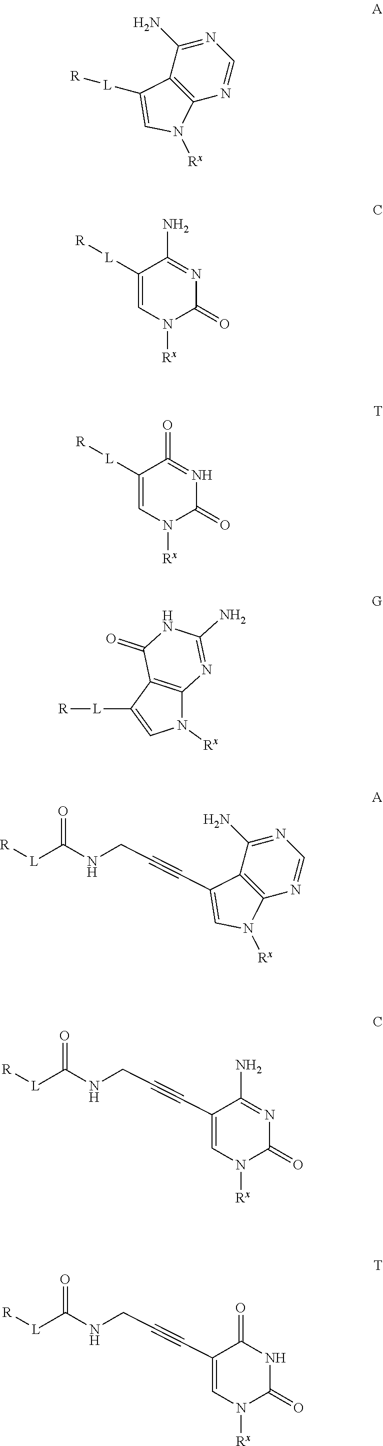

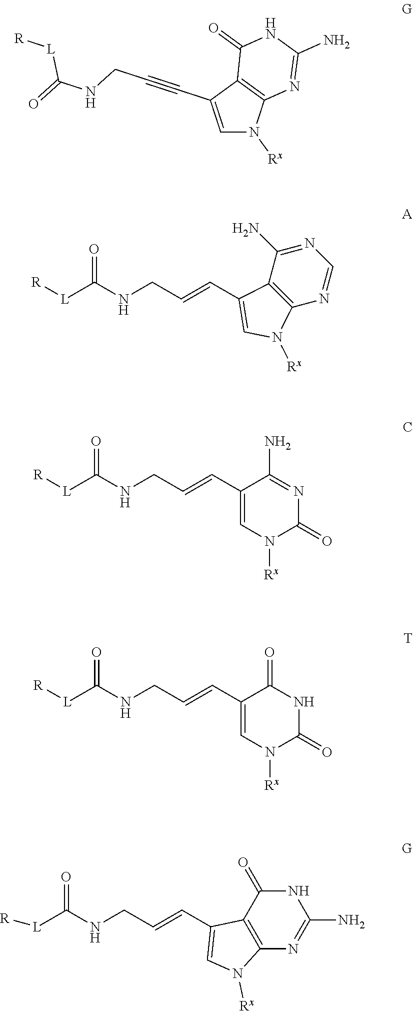

wherein L represents a linker, including a cleavable linker described herein; R^{x} represents a ribose or deoxyribose moiety as described above, or a ribose or deoxyribose moiety with the 5′ position substituted with mono-, di- or tri-phosphates; R represents the first, second or third functional moiety described herein.

In some embodiments, non-limiting exemplary unlabeled nucleotide containing a hapten moiety covalently attached via a cleavable linker are shown below:

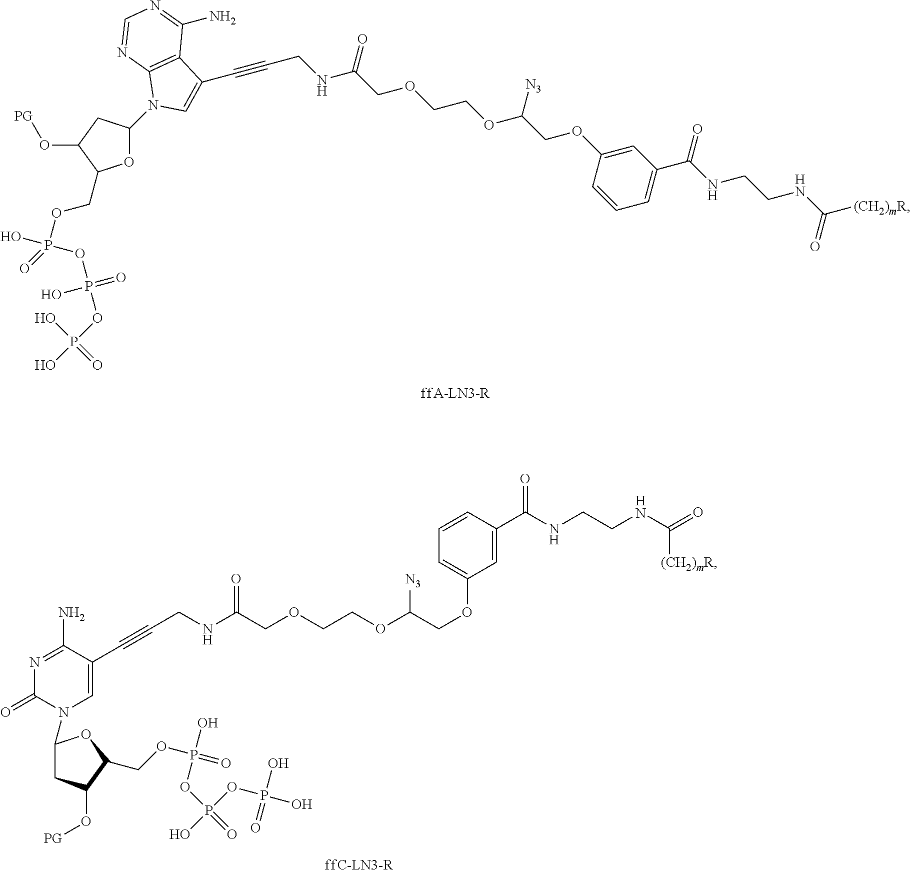

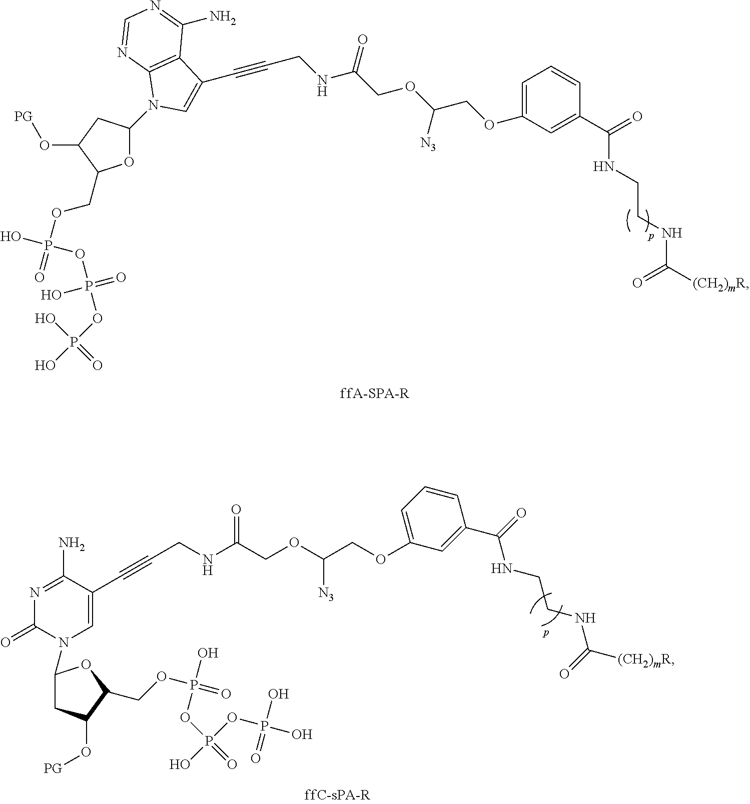

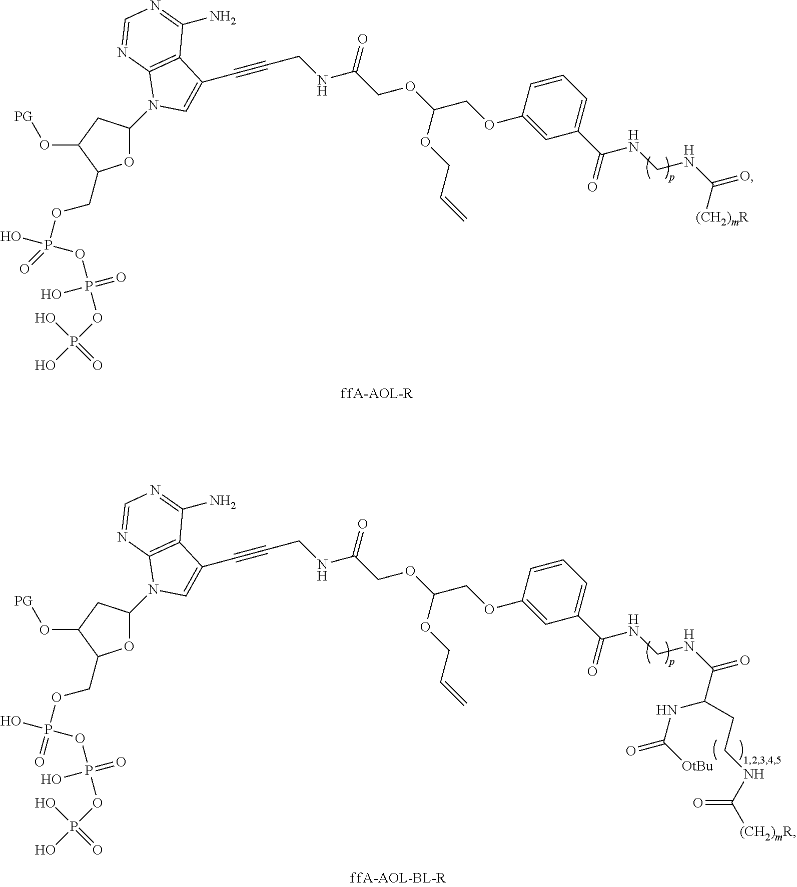

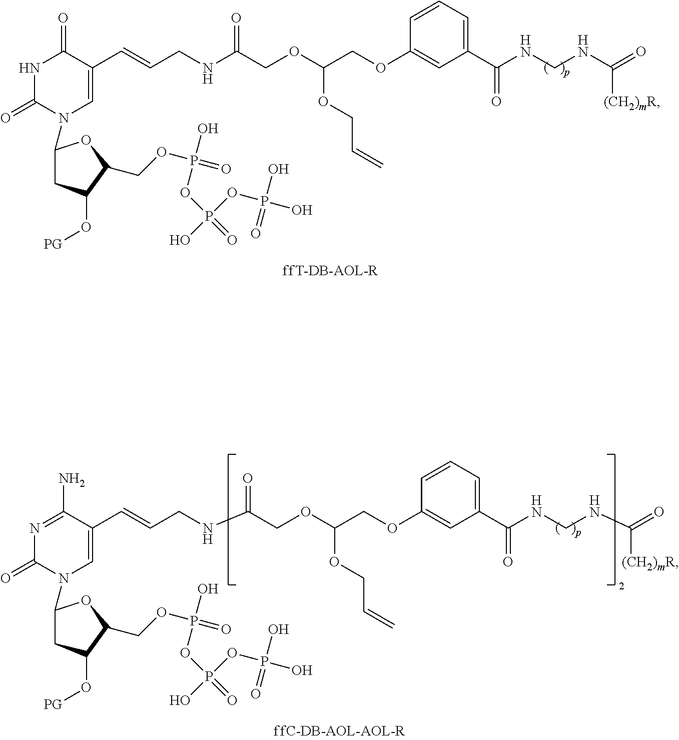

wherein PG stands for the 3′ blocking groups described herein; p is an integer of 1, 2, 3, 4, 5, 6, 7, 8, 9, or 10; and m is 0, 1, 2, 3, 4, or 5. In one embodiment, —O—PG is AOM. In another embodiment, —O—PG is —O-azidomethyl. In one embodiment, m is 5. In another embodiment, m is 0. In another embodiment, m is 2. In some further embodiments, p is 1, 2, 3, 4 or 5.

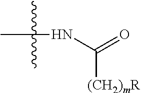

refers to the connection point of the first/second/third functional moiety with the cleavable linker as a result of a reaction between an amino group of the linker moiety and the carboxyl group of the first/second/third functional moiety. In further embodiments, the nucleotide may be attached to the hapten moiety via more than one of the same cleavable linkers (such as LN3-LN3, sPA-SPA, AOL-AOL). In other embodiments, the nucleotide may be attached to the hapten moiety via two or more different cleavable linkers (such sPA-LN3, sPA-SPA-LN3, SPA-LN3-LN3, etc.). In addition, the linker may further include additional PEG spacers as described herein, for example, between R and
(CH₂)ₘ—. In any embodiments of the labeled nucleotide described herein, the nucleotide is a nucleotide triphosphate. In further embodiments, the nucleotide has a 2′ deoxyribose.

### General Description on Sequencing by Synthesis

In a specific embodiment, a synthetic step is carried out and may optionally comprise incubating a template or target polynucleotide strand with a reaction mixture comprising fluorescently labeled nucleotides of the disclosure. A polymerase can also be provided under conditions which permit formation of a phosphodiester linkage between a free 3′ hydroxy group on a polynucleotide strand annealed to the template or target polynucleotide strand and a 5′ phosphate group on the labeled nucleotide. Thus, a synthetic step can include formation of a polynucleotide strand as directed by complementary base pairing of nucleotides to a template/target strand.

In all embodiments of the methods, the detection step may be carried out while the polynucleotide strand into which the labeled nucleotides are incorporated is annealed to a template/target strand, or after a denaturation step in which the two strands are separated. Further steps, for example chemical or enzymatic reaction steps or purification steps, may be included between the synthetic step and the detection step. In particular, the polynucleotide strand incorporating the labeled nucleotide(s) may be isolated or purified and then processed further or used in a subsequent analysis. By way of example, polynucleotide strand incorporating the labeled nucleotide(s) as described herein in a synthetic step may be subsequently used as labeled probes or primers. In other embodiments, the product of the synthetic step set forth herein may be subject to further reaction steps and, if desired, the product of these subsequent steps purified or isolated.

Suitable conditions for the synthetic step will be well known to those familiar with standard molecular biology techniques. In one embodiment, a synthetic step may be analogous to a standard primer extension reaction using nucleotide precursors, including the labeled nucleotides as described herein, to form an extended polynucleotide strand (primer polynucleotide strand) complementary to the template/target strand in the presence of a suitable polymerase enzyme. In other embodiments, the synthetic step may itself form part of an amplification reaction producing a labeled double stranded amplification product comprised of annealed complementary strands derived from copying of the primer and template polynucleotide strands. Other exemplary synthetic steps include nick translation, strand displacement polymerization, random primed DNA labeling, etc. A particularly useful polymerase enzyme for a synthetic step is one that is capable of catalyzing the incorporation of the labeled nucleotides as set forth herein. A variety of naturally occurring or mutant/modified polymerases can be used. By way of example, a thermostable polymerase can be used for a synthetic reaction that is carried out using thermocycling conditions, whereas a thermostable polymerase may not be desired for isothermal primer extension reactions. Suitable thermostable polymerases which are capable of incorporating the labeled nucleotides according to the disclosure include those described in WO 2005/024010 or WO06120433, each of which is incorporated herein by reference. In synthetic reactions which are carried out at lower temperatures such as 37° C., polymerase enzymes need not necessarily be thermostable polymerases, therefore the choice of polymerase will depend on a number of factors such as reaction temperature, pH, strand-displacing activity and the like.

In specific non-limiting embodiments, the disclosure encompasses methods of nucleic acid sequencing, re-sequencing, whole genome sequencing, single nucleotide polymorphism scoring, any other application involving the detection of the modified nucleotide or nucleoside labeled with dyes set forth herein when incorporated into a polynucleotide.

SBS generally involves sequential addition of one or more nucleotides or oligonucleotides to a growing polynucleotide chain in the 5′ to 3′ direction using a polymerase or ligase in order to form an extended polynucleotide chain complementary to the template/target nucleic acid to be sequenced. The identity of the base present in one or more of the added nucleotide(s) can be determined in a detection or “imaging” step. The identity of the added base may be determined after each nucleotide incorporation step. The sequence of the template may then be inferred using conventional Watson-Crick base-pairing rules. The use of the nucleotides labeled with dyes set forth herein for determination of the identity of a single base may be useful, for example, in the scoring of single nucleotide polymorphisms, and such single base extension reactions are within the scope of this disclosure.

In an embodiment of the present disclosure, the sequence of a template/target polynucleotide is determined by detecting the incorporation of one or more nucleotides into a nascent strand complementary to the template polynucleotide to be sequenced through the detection of fluorescent label(s) attached to the incorporated nucleotide(s). Sequencing of the template polynucleotide can be primed with a suitable primer (or prepared as a hairpin construct which will contain the primer as part of the hairpin), and the nascent chain is extended in a stepwise manner by addition of nucleotides to the 3′ end of the primer in a polymerase-catalyzed reaction.

In particular embodiments, each of the different nucleotide triphosphates (A, T, G and C) may be labeled with a unique fluorophore and also comprises a blocking group at the 3′ position to prevent uncontrolled polymerization. Alternatively, one of the four nucleotides may be unlabeled (dark). The polymerase enzyme incorporates a nucleotide into the nascent chain complementary to the template/target polynucleotide, and the blocking group prevents further incorporation of nucleotides. Any unincorporated nucleotides can be washed away and the fluorescent signal from each incorporated nucleotide can be “read” optically by suitable means, such as a charge-coupled device using light source excitation and suitable emission filters. The 3′-blocking group and fluorescent dye compounds can then be removed (deprotected) (simultaneously or sequentially) to expose the nascent chain for further nucleotide incorporation.

The method, as exemplified above, utilizes the incorporation of fluorescently labeled, 3′ blocked nucleotides A, G, C, and T into a growing strand complementary to the immobilized polynucleotide, in the presence of DNA polymerase. The polymerase incorporates a base complementary to the target polynucleotide but is prevented from further addition by the 3′ blocking group. The label of the incorporated nucleotide can then be determined, and the blocking group removed by chemical cleavage to allow further polymerization to occur. The nucleic acid template to be sequenced in a SBS reaction may be any polynucleotide that it is desired to sequence. The nucleic acid template for a sequencing reaction will typically comprise a double stranded region having a free 3′ hydroxy group that serves as a primer or initiation point for the addition of further nucleotides in the sequencing reaction. The region of the template to be sequenced will overhang this free 3′ hydroxy group on the complementary strand. The overhanging region of the template to be sequenced may be single stranded but can be double-stranded, provided that a “nick” is present on the strand complementary to the template strand to be sequenced to provide a free 3′ OH group for initiation of the sequencing reaction. In such embodiments, sequencing may proceed by strand displacement. In certain embodiments, a primer bearing the free 3′ hydroxy group may be added as a separate component (e.g., a short oligonucleotide) that hybridizes to a single-stranded region of the template to be sequenced. Alternatively, the primer and the template strand to be sequenced may each form part of a partially self-complementary nucleic acid strand capable of forming an intra-molecular duplex, such as for example a hairpin loop structure. Hairpin polynucleotides and methods by which they may be attached to solid supports are disclosed in PCT Publication Nos. WO0157248 and WO2005/047301, each of which is incorporated herein by reference. Nucleotides can be added successively to a growing primer, resulting in synthesis of a polynucleotide chain in the 5′ to 3′ direction. The nature of the base which has been added may be determined, particularly but not necessarily after each nucleotide addition, thus providing sequence information for the nucleic acid template. Thus, a nucleotide is incorporated into a nucleic acid strand (or polynucleotide) by joining of the nucleotide to the free 3′ hydroxy group of the nucleic acid strand via formation of a phosphodiester linkage with the 5′ phosphate group of the nucleotide.

The nucleic acid template to be sequenced may be DNA or RNA, or even a hybrid molecule comprised of deoxynucleotides and ribonucleotides. The nucleic acid template may comprise naturally occurring and/or non-naturally occurring nucleotides and natural or non-natural backbone linkages, provided that these do not prevent copying of the template in the sequencing reaction.

In certain embodiments, the nucleic acid template to be sequenced may be attached to a solid support via any suitable linkage method known in the art, for example via covalent attachment. In certain embodiments template polynucleotides may be attached directly to a solid support (e.g., a silica-based support). However, in other embodiments of the disclosure the surface of the solid support may be modified in some way so as to allow either direct covalent attachment of template polynucleotides, or to immobilize the template polynucleotides through a hydrogel or polyelectrolyte multilayer, which may itself be non-covalently attached to the solid support.

Arrays in which polynucleotides have been directly attached to a support (for example, silica-based supports such as those disclosed in WO00/06770 (incorporated herein by reference), wherein polynucleotides are immobilized on a glass support by reaction between a pendant epoxide group on the glass with an internal amino group on the polynucleotide. In addition, polynucleotides can be attached to a solid support by reaction of a sulfur-based nucleophile with the solid support, for example, as described in WO2005/047301 (incorporated herein by reference). A still further example of solid-supported template polynucleotides is where the template polynucleotides are attached to hydrogel supported upon silica-based or other solid supports, for example, as described in WO00/31148, WO01/01143, WO02/12566, WO03/014392, U.S. Pat. No. 6,465,178 and WO00/53812, each of which is incorporated herein by reference.

A particular surface to which template polynucleotides may be immobilized is a polyacrylamide hydrogel. Polyacrylamide hydrogels are described in the references cited above and in WO2005/065814, which is incorporated herein by reference. Specific hydrogels that may be used include those described in WO2005/065814 and U.S. Pub. No. 2014/0079923. In one embodiment, the hydrogel is PAZAM (poly(N-(5-azidoacetamidylpentyl) acrylamide-co-acrylamide)).

DNA template molecules can be attached to beads or microparticles, for example, as described in U.S. Pat. No. 6,172,218 (which is incorporated herein by reference). Attachment to beads or microparticles can be useful for sequencing applications. Bead libraries can be prepared where each bead contains different DNA sequences. Exemplary libraries and methods for their creation are described in Nature, 437, 376-380 (2005); Science, 309, 5741, 1728-1732 (2005), each of which is incorporated herein by reference. Sequencing of arrays of such beads using nucleotides set forth herein is within the scope of the disclosure.

Template(s) that are to be sequenced may form part of an “array” on a solid support, in which case the array may take any convenient form. Thus, the method of the disclosure is applicable to all types of high-density arrays, including single-molecule arrays, clustered arrays, and bead arrays. Nucleotides labeled with dye compounds of the present disclosure may be used for sequencing templates on essentially any type of array, including but not limited to those formed by immobilization of nucleic acid molecules on a solid support.

However, nucleotides labeled with dye compounds of the disclosure are particularly advantageous in the context of sequencing of clustered arrays. In clustered arrays, distinct regions on the array (often referred to as sites, or features) comprise multiple polynucleotide template molecules. Generally, the multiple polynucleotide molecules are not individually resolvable by optical means and are instead detected as an ensemble. Depending on how the array is formed, each site on the array may comprise multiple copies of one individual polynucleotide molecule (e.g., the site is homogenous for a particular single- or double-stranded nucleic acid species) or even multiple copies of a small number of different polynucleotide molecules (e.g., multiple copies of two different nucleic acid species). Clustered arrays of nucleic acid molecules may be produced using techniques generally known in the art. By way of example, WO 98/44151 and WO00/18957, each of which is incorporated herein, describe methods of amplification of nucleic acids wherein both the template and amplification products remain immobilized on a solid support in order to form arrays comprised of clusters or “colonies” of immobilized nucleic acid molecules. The nucleic acid molecules present on the clustered arrays prepared according to these methods are suitable templates for sequencing using nucleotides labeled with dye compounds of the disclosure.

Nucleotides labeled with dye compounds of the present disclosure are also useful in sequencing of templates on single molecule arrays. The term “single molecule array” or “SMA” as used herein refers to a population of polynucleotide molecules, distributed (or arrayed) over a solid support, wherein the spacing of any individual polynucleotide from all others of the population is such that it is possible to individually resolve the individual polynucleotide molecules. The target nucleic acid molecules immobilized onto the surface of the solid support can thus be capable of being resolved by optical means in some embodiments. This means that one or more distinct signals, each representing one polynucleotide, will occur within the resolvable area of the particular imaging device used.

Single molecule detection may be achieved wherein the spacing between adjacent polynucleotide molecules on an array is at least 100 nm, more particularly at least 250 nm, still more particularly at least 300 nm, even more particularly at least 350 nm. Thus, each molecule is individually resolvable and detectable as a single molecule fluorescent point, and fluorescence from said single molecule fluorescent point also exhibits single step photobleaching.

The terms “individually resolved” and “individual resolution” are used herein to specify that, when visualized, it is possible to distinguish one molecule on the array from its neighboring molecules. Separation between individual molecules on the array will be determined, in part, by the particular technique used to resolve the individual molecules. The general features of single molecule arrays will be understood by reference to published applications WO00/06770 and WO 01/57248, each of which is incorporated herein by reference. Although one use of the labeled nucleotides of the disclosure is in SBS reactions, the utility of such nucleotides is not limited to such methods. In fact, the labeled nucleotides described herein may be used advantageously in any sequencing methodology which requires detection of fluorescent labels attached to nucleotides incorporated into a polynucleotide.

In particular, nucleotides labeled with dye compounds of the disclosure may be used in automated fluorescent sequencing protocols, particularly fluorescent dye-terminator cycle sequencing based on the chain termination sequencing method of Sanger and co-workers. Such methods generally use enzymes and cycle sequencing to incorporate fluorescently labeled dideoxynucleotides in a primer extension sequencing reaction. So-called Sanger sequencing methods, and related protocols (Sanger-type), utilize randomized chain termination with labeled dideoxynucleotides.

### EXAMPLES

Additional embodiments are disclosed in further detail in the following examples, which are not in any way intended to limit the scope of the claims.

### Example 1. Synthesis of Dye A1-DBCO and NR550C4-DBCO

### 1A. Synthesis of NR550C4-DBCO

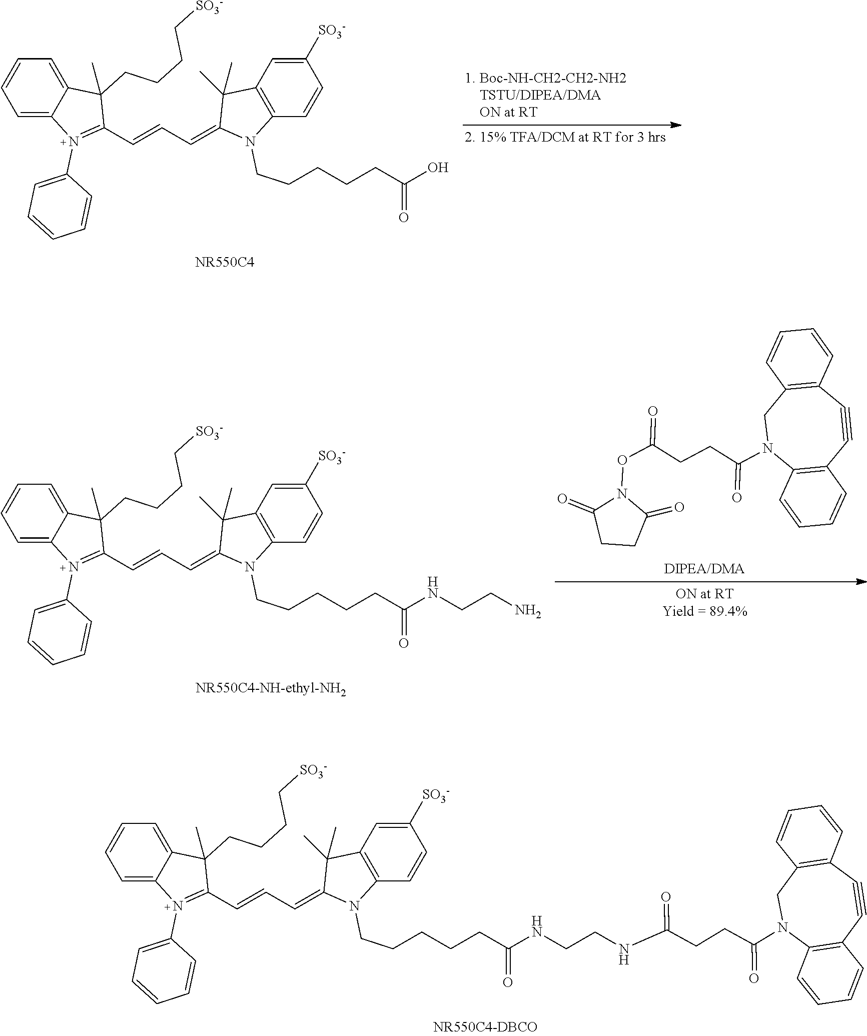

Synthesis of NR550C4-NH-ethyl-NH₂: In a round-bottom flask, NR550C4 dye (1 eq) was dissolved in DMF (anhydrous, 1 mL) and evaporated to dry. The procedure was repeated for 3 times. Anhydrous DMA and Hunig's base (10 equivalents) were then pipetted into the round bottom flask. Coupling agent TSTU (2 equivalents) was added in one portion. The reaction mixture was kept at room temperature at N₂ atmosphere. After 30 min, TLC analysis indicated that the reaction completed. N-Boc-ethylenediamine (5 eq) in 0.1 M TEAB was added to the reaction mixture and stirred at room temperature for 16 h. TLC showed complete consumption of the activated ester. The reaction was quenched with TEAB buffer (0.1M, 10 ml) purified by reverse phase C18 column eluted with gradient of solvent water and CH₃CN. Intermediate product (NR550C4-NH-ethyl-NH-Boc) fractions were combined and dried under vacuum to remove the solvent. Then the NR550C4-NH-ethyl-NH-Boc was dissolved in 8.5 mL of DCM and 1.5 mL of TFA was added. The mixture was reacted at room temperature for 3 hours and the solvent was removed under reduced pressure. The compound was purified by reverse phase C18 column (Biotage) eluted with gradient of solvent water and CH₃CN to obtain desired compound with 77% yield. Found m/z for [M-H]− (LC-MS) 761.60.

Synthesis of NR550C4-DBCO: In a 10 ml round-bottom flask, DBCO-NHS ester (30 μmol, 3 eq) was dissolved in anhydrous DMA (2 mL), DIEPA (10 eq, 100 μmol) was then added into the round bottom flask. Then a solution of NR550C4-NH-ethyl-NH₂ (10 μmol in 0.2 ml H₂O, 1 eq) was added to the reaction mixture and stirred at room temperature over-night. The reaction was quenched with TEAB buffer (0.1M, 20 ml) and purified sing a C18 reverse phase column (Biotage) eluted with gradient of solvent water and CH₃CN to obtain desired compound with 89.9% yield. Found m/z for [M-H]− (LC-MS) 1048.54.

### 1B. Synthesis of Dye A1-DBCO

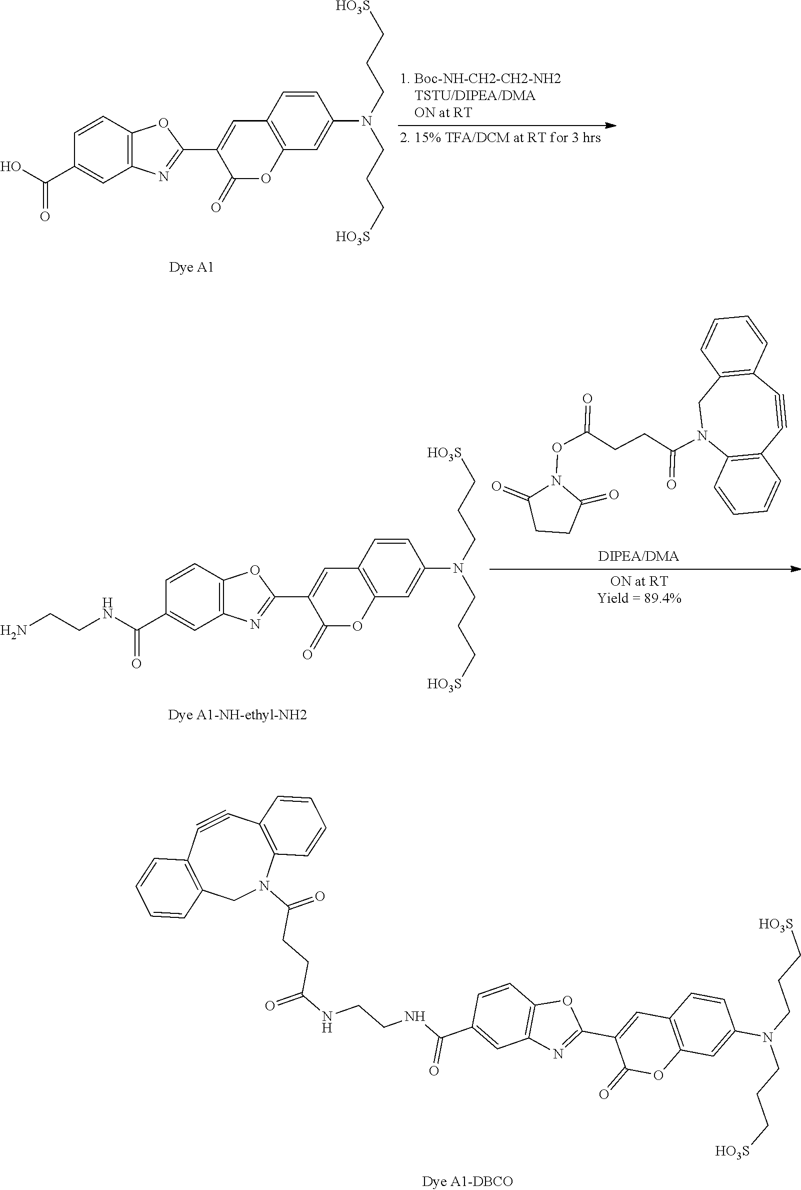

Dye A1-DBCO was synthesized following a procedure similar to the preparation of NR550C4-DBCO. Yield=79% and m/z for [M-H]− (LC-MS)=895.34.

### Example 2, Synthesis of ffC-G2-AOL-TCO, ffC-(AOL)₂-TCO, ffC-G2-AOL-DBCO and ffC-G2-AOL-PEG4-DBCO

### 2A. Synthesis of ffC-G2-AOL-DBCO

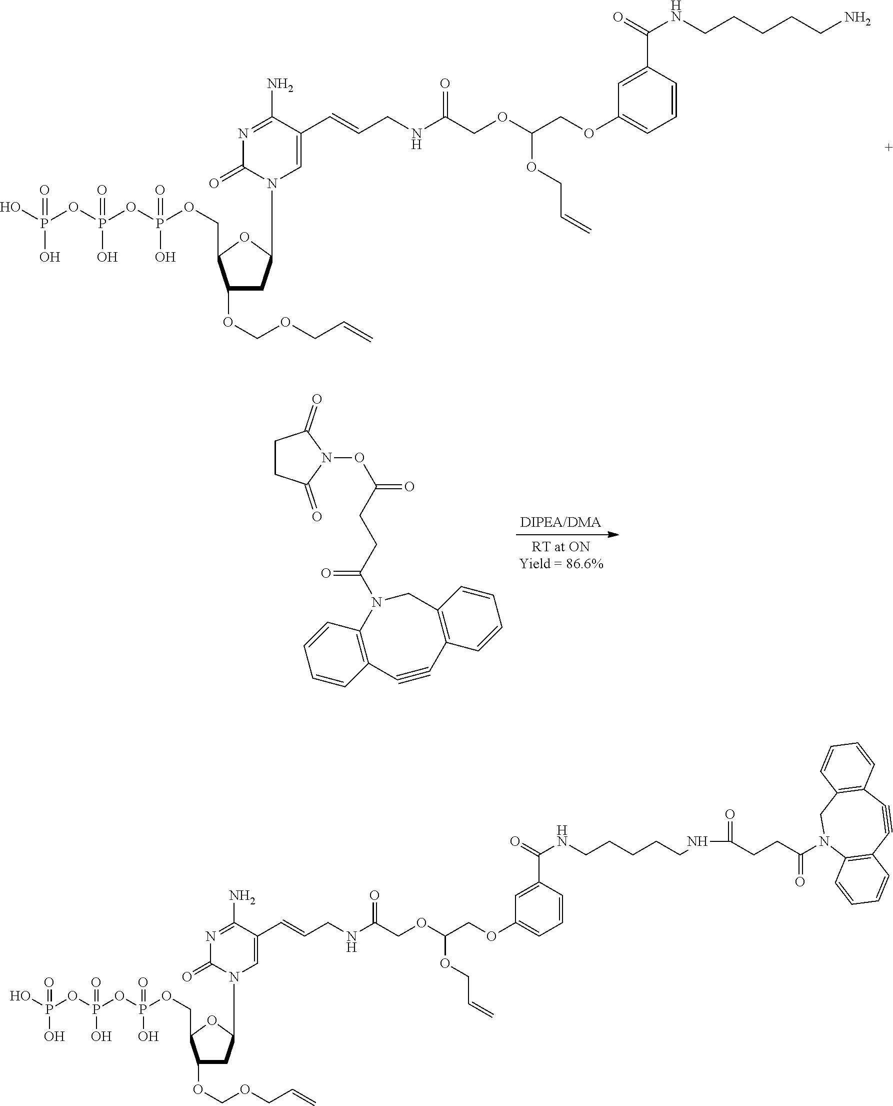

In a 10 ml round-bottom flask, DBCO-NHS ester (31.2 μmol, 3 eq) was dissolved in anhydrous DMA (2 mL), DIEPA (10 eq, 104 μmol) was then added into the round bottom flask. Then a solution of pppC-G2-AOL (10.4 μmol in 0.2 ml H₂O, 1 eq) was added to the reaction mixture and stirred at room temperature over-night. TLC showed complete consumption of the activated ester. The reaction was quenched with TEAB buffer (0.1M, 20 ml) and loaded on a DEAE Sephadex column (25 g Biotage column). The column was eluted with gradient as below. A: 0.1 M TEAB buffer (10% CH₃CN) B: 1 M TEAB buffer (10% CH₃CN). The product peak was collected and desalted using a C₁₈ reverse phase column eluted with gradient of solvent water and CH₃CN. The desalted was further purified by preparative HPLC using a with gradient of solvent 0.1 M TEAB and CH₃CN to obtain desired compound with 86.6% yield. Found m/z for [M-H]− (LC-MS) 1241.42.

### 2B. Synthesis of ffC-G2-AOL-TCO

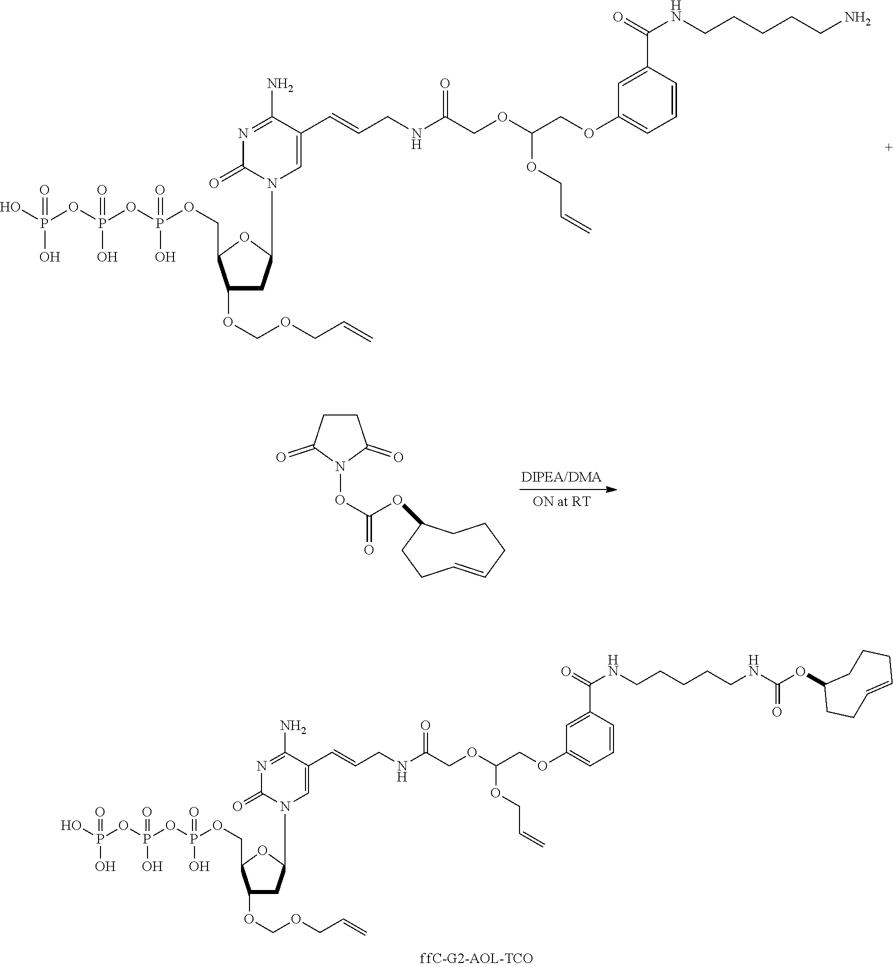

ffC-G2-AOL-TCO was synthesized following a procedure similar to the preparation of ffC-G2-AOL-DBCO. Yield=35.5% and m/z for [M-H]− (LC-MS)=1105.58.

### 2C. Synthesis of ffC-G2-AOL-PEG4-DBCO

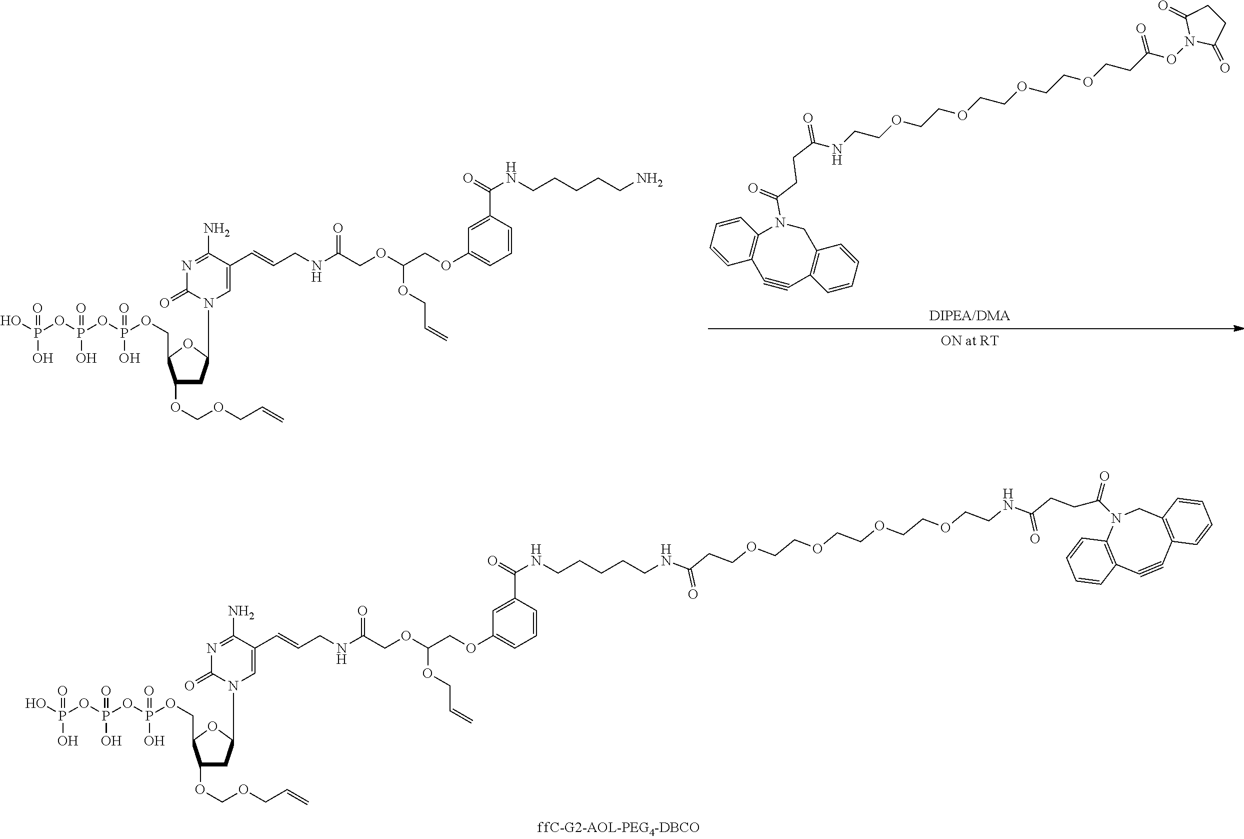

ffC-G2-AOL-PEG4-DBCO was synthesized following a procedure similar to the preparation of ffC-G2-AOL-DBCO. Yield=15% and m/z for [M-H]− (LC-MS)=1487.75.

### 2D. Synthesis of ffA-G2-AOL-TCO

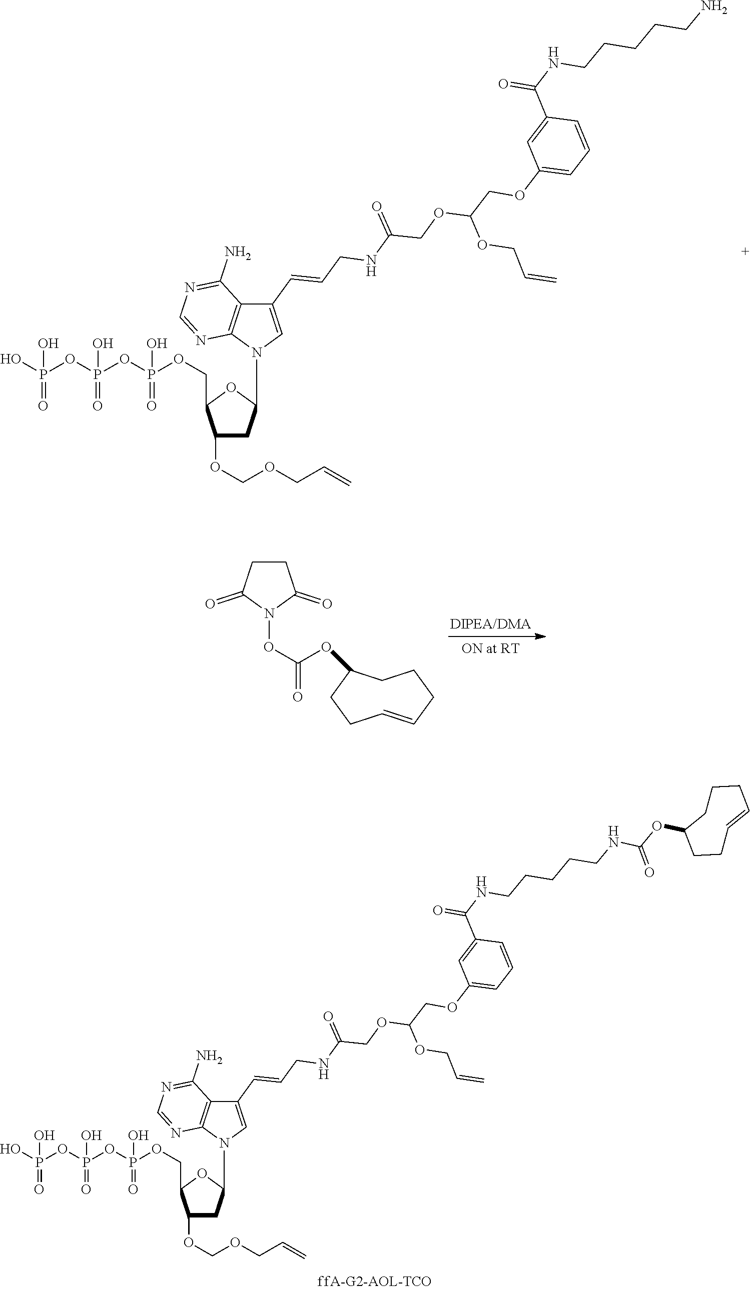

ffC-G2-AOL-TCO was synthesized following a procedure similar to the preparation of ffC-G2-AOL-DBCO. Yield=80% and m/z for [M-H]− (LC-MS)=1128.51.

### 2E, Synthesis of ftT-G2-AOL-DBCO

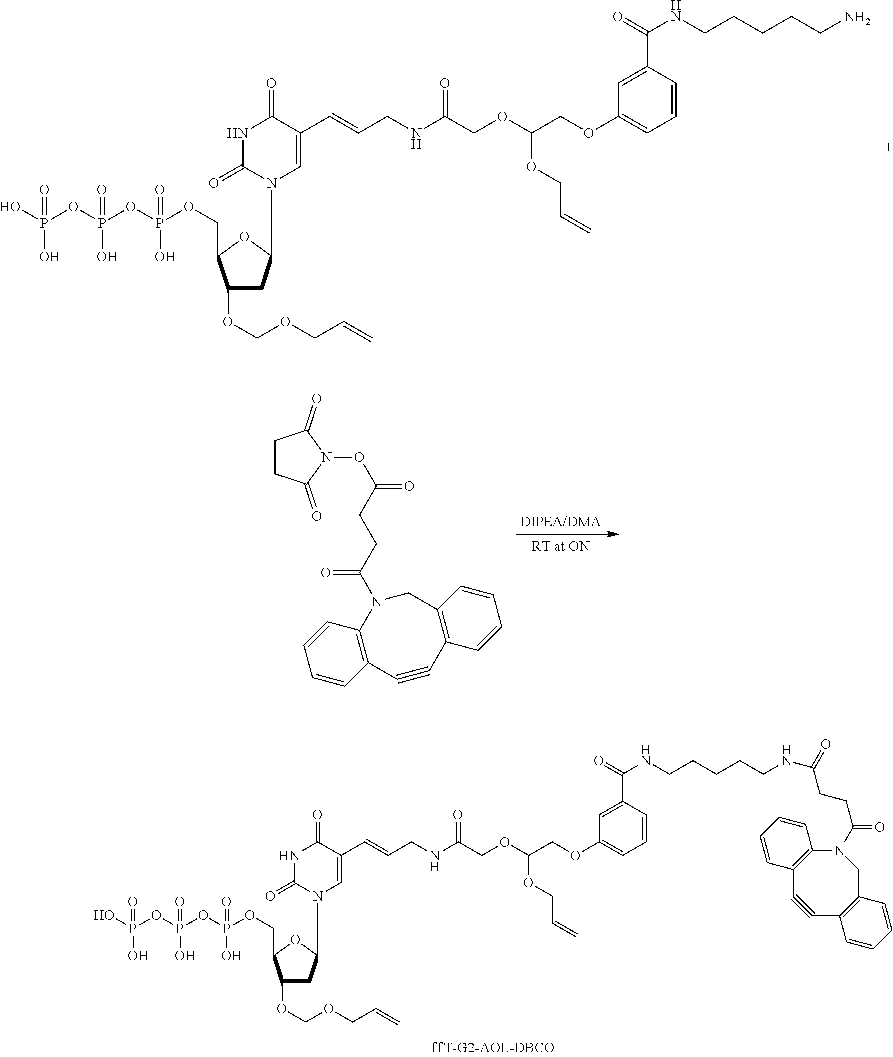

ffT-G2-AOL-TCO was synthesized following a procedure similar to the preparation of ffC-G2-AOL-TCO. Yield=71.4% and m/z for [M-H]− (LC-MS)=1241.98.

### 2F. Synthesis of ffC-G2-AOL-AOL-TCO

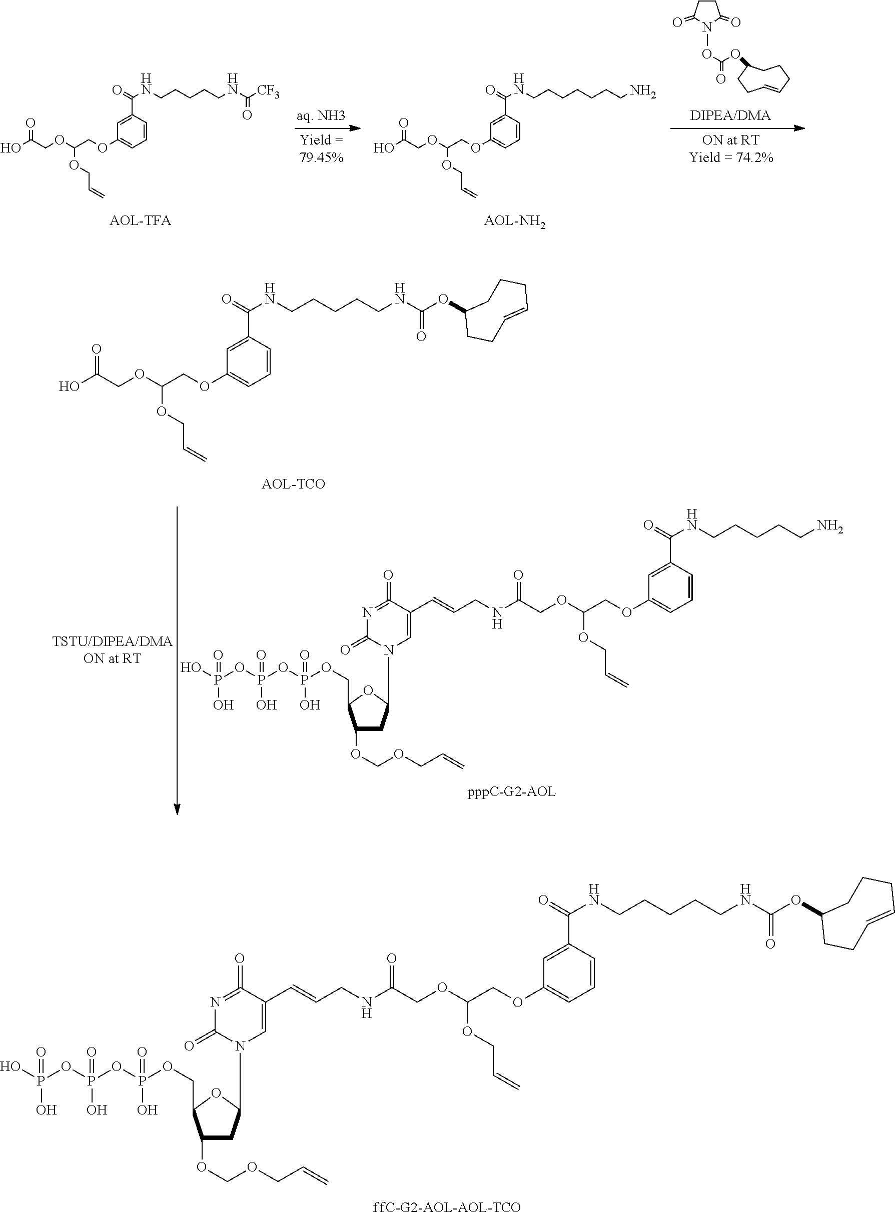

Synthesis of AOL-NH₂: In a 25 mL round bottomed flask, AOL-TFA (200 μmol) was dissolved in 2.1 mL of methanol and 7.52 mL of aqueous ammonia (35%) and stirred overnight at room temperature. The volatiles were removed, and the crude was purified with flash column chromatography using a reverse phase C₁₈ column. Yield=79.4% and m/z for [M-H]− (LC-MS)=379.17.

Synthesis of AOL-TCO: In a round-bottom flask, TCO-NHS (1 eq, 42.6 μmol) was dissolved in 4 mL of anhydrous DMA and Hunig's base (5 eq, 213 μmol) were then pipetted into the round bottom flask. The reaction mixture was kept at room temperature at N₂ atmosphere and AOL-NH₂ (2 eq, 85.2 μmol) in DMA was added to the reaction mixture and stirred at room temperature for 16 h. TLC showed complete consumption of the activated ester and a red spot appeared below the activated ester. The reaction was quenched with water purified by reverse phase C18 column eluted with gradient of solvent water and CH₃CN to obtain desired compound with 74.2% yield. Found m/z for [M-H]− (LC-MS) 531.36.

Synthesis of ffC-G2-AOL-AOL-TCO: In a 10 ml round-bottom flask, AOL-TCO (31.9 μmol, 1 eq) was dissolved in DMF (anhydrous, 1 mL) and evaporated to dry. The procedure was repeated for 3 times. Anhydrous DMA (3 mL) and Hunig's base (10 eq, 319 μmol) were then pipetted into the round bottom flask. Coupling agent TSTU (1.5 eq, 47.8 μmol) was added in one portion. The reaction mixture was kept at room temperature at N₂ atmosphere. After 30 min, TLC analysis indicated that the reaction completed. Then a solution of pppC-G2-AOL (47.8 μmol in 0.3 ml 0.1 M TEAB, 1.5 eq) was added to the reaction mixture and stirred at room temperature over-night. TLC showed complete consumption of the activated ester. The reaction was quenched with TEAB buffer (0.1M, 30 ml) and loaded on a DEAE Sephadex column (25 g Biotage column). The column was eluted with gradient as below. A: 0.1 M TEAB buffer (10% CH₃CN) B: 1 M TEAB buffer (10% CH₃CN). The product peak was collected and desalted using a C18 reverse phase column eluted with gradient of solvent water and CH₃CN. The desalted was further purified by preparative HPLC using a with gradient of solvent 0.1 M TEAB and CH₃CN to obtain desired compound with 65% yield. Found m/z for [M-H]− (LC-MS) 1467.47.

### Example 3. Synthesis of ffC-G2-AOL-PEG₄-N₃

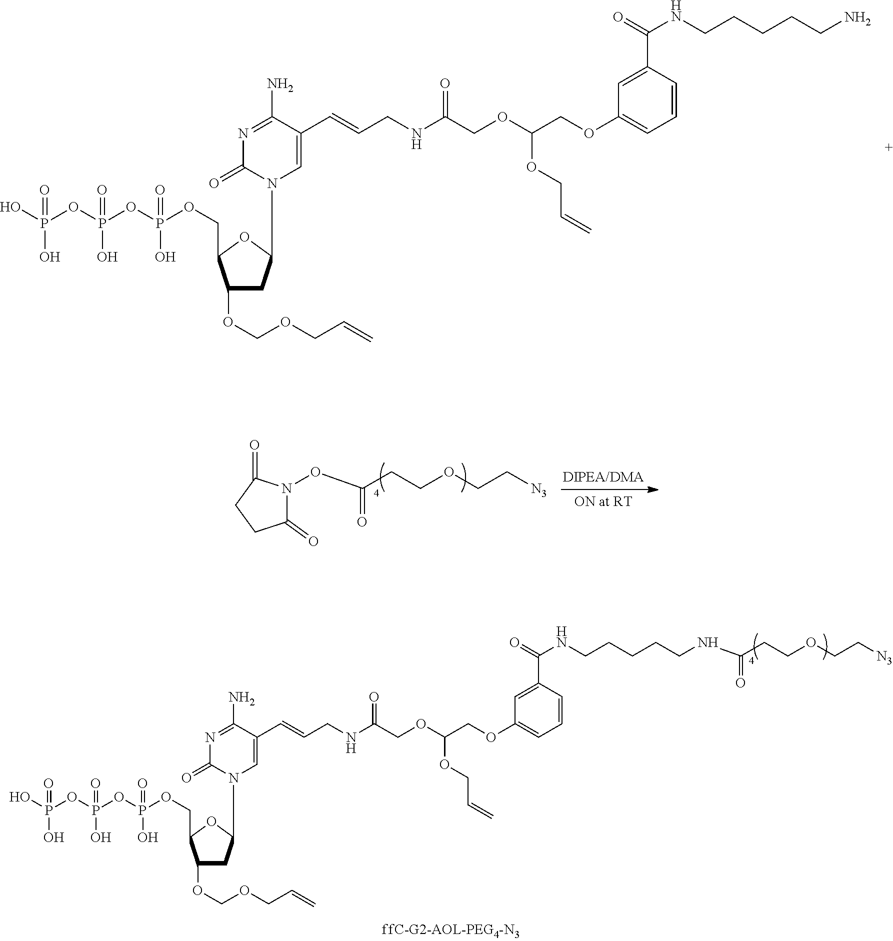

ffC-G2-AOL-PEG₄-N₃ was synthesized following a procedure that is similar to the preparation of ffC-G2-AOL-DBCO. Yield=28% and m/z for [M]⁻ (LC-MS)=1226.42.

### Example 4. Synthesis of Dye A1-Tz, Dye A1-PEG₃-N₃, Dye A1-PEG₁₀-N₃

### 4A. Synthesis of Dye A1-Tz

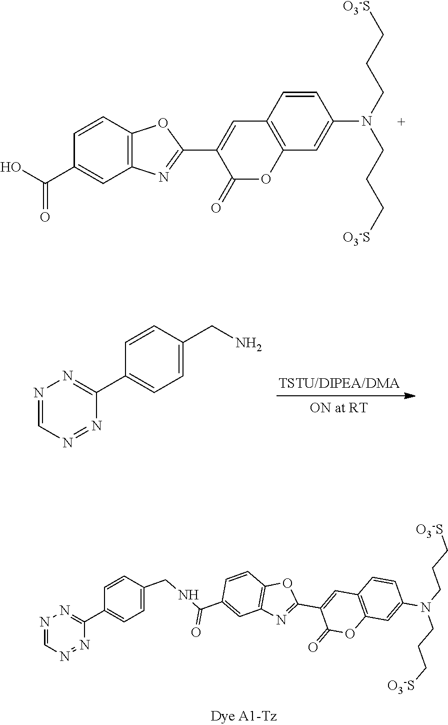

In a round-bottom flask, Dye A1 (1 eq, 74.5 μmol, 42 mg) was dissolved in DMF (anhydrous, 1 mL) and evaporated to dry. The procedure was repeated for 3 times. Anhydrous DMF and Hunig's base (5 eq, 372.6 μmol, 64.9 μL) were then pipetted into the round bottom flask. Coupling agent TSTU (1.5 eq, 111.8 μmol, 33.7 mg) was added in one portion. The reaction mixture was kept at room temperature at N₂ atmosphere. After 30 min, TLC analysis indicated that the reaction completed. Tetrazine amine (3 eq, 223.6 μmol, 50 mg) in 0.1 M TEAB was added to the reaction mixture and stirred at room temperature for 16 h. TLC showed complete consumption of the activated ester. The reaction was quenched with water purified by reverse phase C18 column eluted with gradient of solvent 0.05% TFA in water and CH₃CN to obtain desired compound with 16% yield. Found m/z for [M-H]− (LC-MS) 734.23.

### 4B. Synthesis of Dye A1-PEG₃-N₃

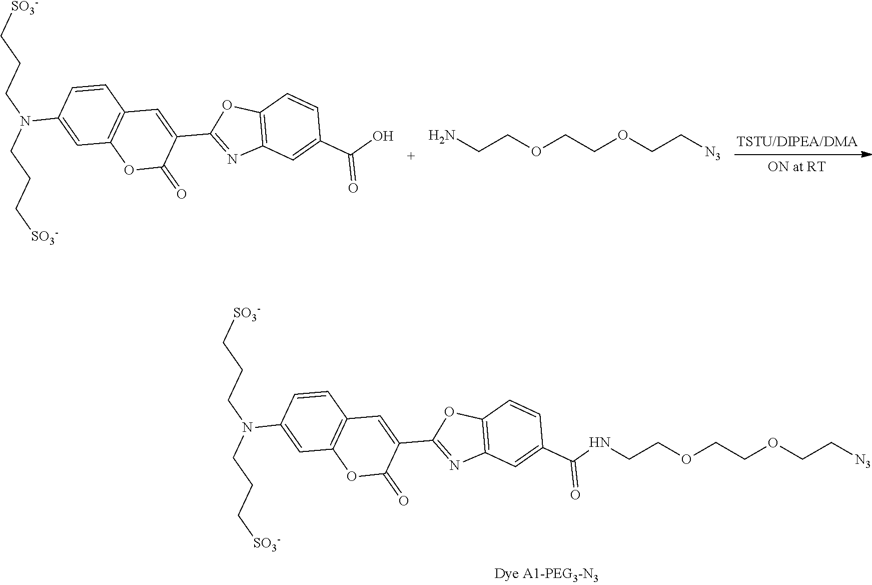

In a round-bottom flask, Dye A1 (1 eq) was dissolved in DMF (anhydrous, 1 mL) and evaporated to dry. The procedure was repeated for 3 times. Anhydrous DMA and Hunig's base (10 equivalents) were then pipetted into the round bottom flask. Coupling agent TSTU (2 equivalents) was added in one portion. The reaction mixture was kept at room temperature at N₂ atmosphere. After 30 min, TLC analysis indicated that the reaction completed. Azido-PEG3-amine (5 eq) in 0.1 M TEAB was added to the reaction mixture and stirred at room temperature for 16 h. TLC showed complete consumption of the activated ester. The reaction was quenched with 0.1 M TEAB and purified by reverse phase C18 column eluted with gradient of solvent water and CH₃CN to obtain desired compound with 54% yield. Found m/z for [M-H]−(LC-MS) 721.46.

### 4C. Dye A-PEG₁₀-N₃

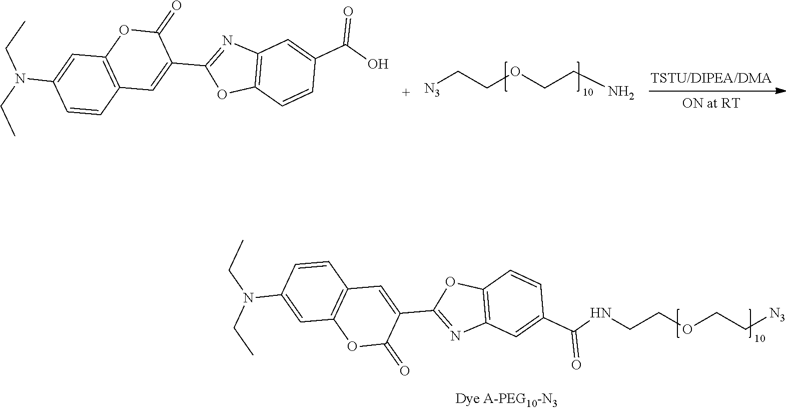

Dye A-PEG₁₀-N₃ was synthesized following a procedure similar to the preparation of Dye A1-PEG₃-N₃. Yield=90% and m/z for [M-H]− (LC-MS)=885.71.

### Example 5. Synthesis of NR550C4-Py-Tz

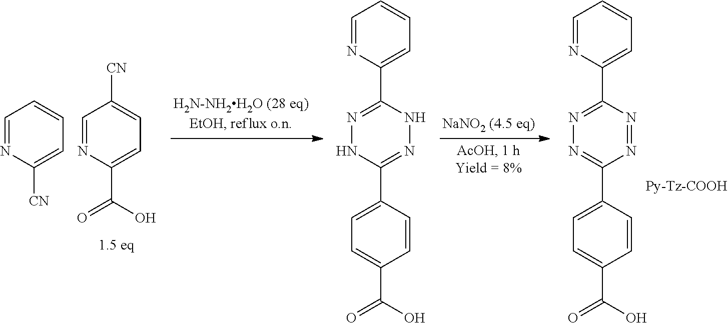

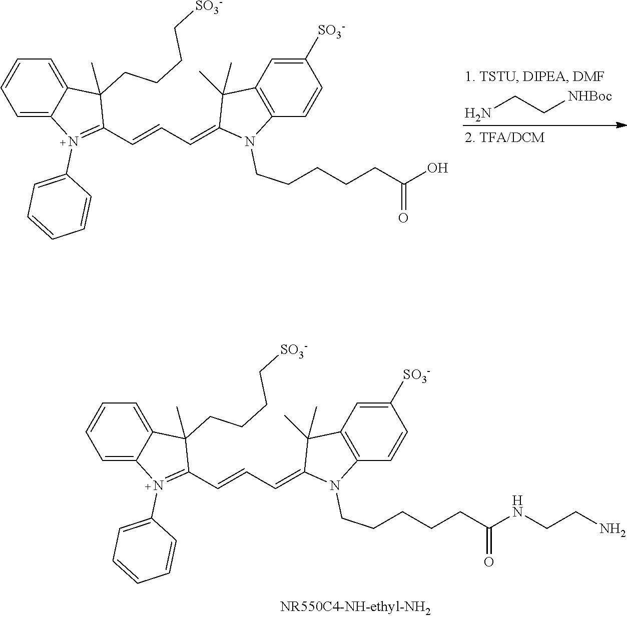

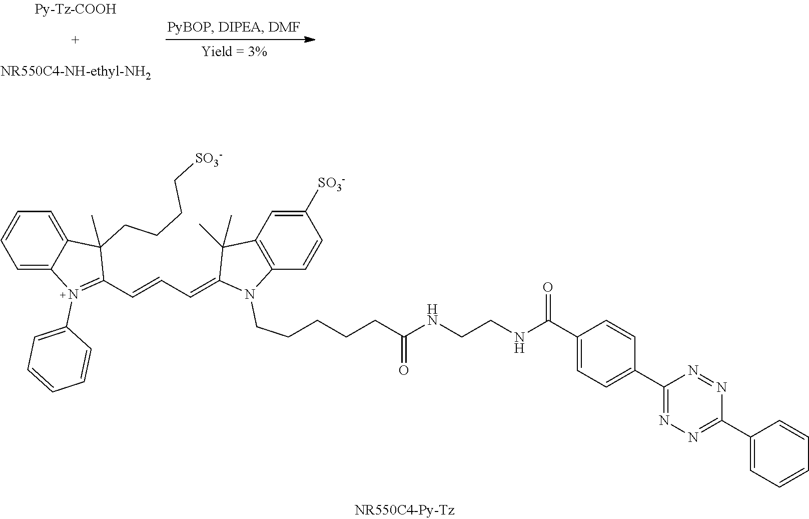

Synthesis of Py-Tz-COOH: In a two neck round bottomed flask, 2-cyanopridine (1 eq, 4.5 μmol, 0.468 g) and 4-cyanobenzoic acid (1.5 eq, 6.75 μmol, 0.993 g) were dissolved in 25 mL of ethanol. Hydrazine monohydrate (28 eq, 126 μmol, 6.1 mL) was added to the above mixture and the reaction mixture was heated at 81 C for 16 hours. The reaction was cooled to room temperature and the orange solid was collected by vacuum filtration. The resulting solid was dissolved in 7 mL of glacial acetic acid and NaNO₂ (4.5 eq, 20.25 μmol, 1.40 g) in 2 mL of water was added at 0° C. The reaction mixture was warmed to room temperature and stirred at room temperature for 1 hour. The purple precipitate formed was filtered and washed with water. The resulting solid was washed with hot DMF, and filtrate was evaporated to remove DMF to yield Py-Tz-COOH as a purple solid (Yield 8%).

Synthesis of NR550C4-Py-Tz: In a round-bottom flask, Py-Tz-COOH (1 eq, 70.2 μmol, 19.6 mg) was dissolved in DMF (anhydrous, 1 mL) and evaporated to dry. The procedure was repeated for 3 times. Anhydrous DMF (6 mL) and Hunig's base (10 eq, 701.9 μmol, 0.122 mL) were then pipetted into the round bottom flask. Coupling agent PyBop (1.2 eq, 84.2 μmol, 43.8 mg) was added in one portion. The reaction mixture was kept at room temperature at N₂ atmosphere. After 30 min, NR550C4-NH-ethyl-NH₂ (2 eq, 140.4 μmol, 106.9 mg) in DMF was added to the reaction mixture and stirred at room temperature for 16 h. The reaction was quenched with 0.1 M TEAB and purified by reverse phase C18 column eluted with gradient of solvent 0.1% TFA in water and CH₃CN to yield NR550C4-Py-Tz (Yield 3%). Found m/z for [M-H]⁻ (LC-MS) 1022.79.

### Example 6. Sequencing by Synthesis (SBS) Experiment on Illumina MiSeq® Instrument

In this experiment, SBS runs were conducted on Illumina MiSeq® instrument with blue and green LED operating at 460 nm and 532 nm. Images were taken simultaneously through collection channels which are in blue (472-520 nm) and green (540-640 nm).

The standard incorporation mixture include: (1) a set of nucleotides comprising dark ffG, ffC-G2-AOL-Dye A, ffA-G2-AOL-BL-NR550S0, ffA-G2-AOL-BL-Dye A, ffT-G2-AOL-Dye D, each comprising a 3′ AOM blocking group; (2) DNA polymerase Pol A; and (3) a glycine buffer. In comparison, the incorporation mix used in the post incorporation labeling method included dark ffG, azide-linked ffC (ffC-G2-AOL-PEG₄-N₃), ffA-G2-AOL-BL-NR550S0, ffA-G2-AOL-BL-Dye A, and ffT-G2-AOL-Dye D, each comprising a 3′ AOM blocking group. Post incorporation reagent contains Dye A1-DBCO and NR550C4-DBCO in 10 mM pH 8 Tris buffer. The Post incorporation mixture was introduced to incorporated ffNs and allowed to bind at 60° C. flush through the flow cell without incubation (2.5 s contact time).

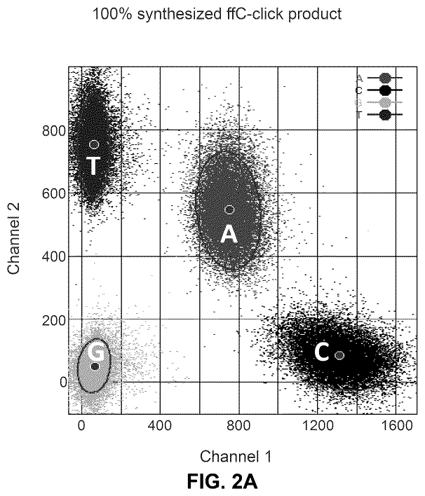

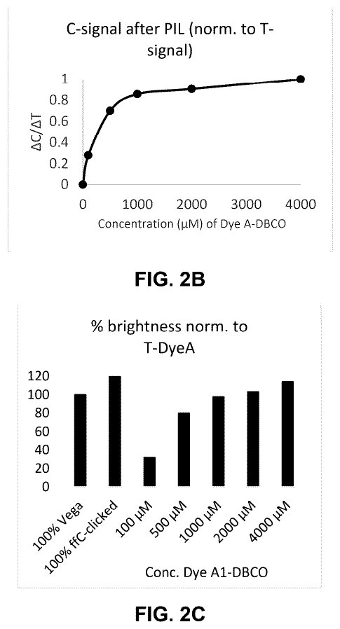

FIG. 2A is a scatterplot of cycle 8 of a blue/green two-channel SBS run conducted with an incorporation mixture containing a set of nucleotides including an unlabeled C nucleotide with azido moiety, and a post incorporation labeling mixture containing a blue coumarin dye A1 functionalized with a dibenzocyclooctyne (DBCO) moiety at 2 μM.

FIG. 2B is a line chart illustrating normalized C nucleotide signal as a function of the concentration of a post incorporation labeling mixture containing a blue coumarin dye (A1-DBCO).

FIG. 2C is a bar chart illustrating the normalized percentage (%) brightness of C nucleotide signal (normalized to ffT labeled with Dye A) of an incorporation mixture containing a labeled fully functionalized C nucleotide (ffC), as compared to an incorporation mixture containing unlabeled C nucleotide with azido moiety, and a post incorporation labeling mixture containing a blue coumarin dye A1-DBCO at various concentrations. An improvement in % brightness was observed when standard labeled ffC was replaced by unlabeled azido modified C nucleotide.

### Example 7. Sequencing by Synthesis (SBS) Experiment on Illumina MiSeq® Instrument

In this experiment, SBS runs were conducted on Illumina MiSeq® instrument with blue and green LED operating at 460 nm and 532 nm. Images were taken simultaneously through collection channels which are in blue (472-520 nm) and green (540-640 nm).

The standard incorporation mixture include: (1) a set of nucleotides comprising dark ffG, ffC-G2-AOL-Dye A, ffA-G2-AOL-BL-NR550S0, ffA-G2-AOL-BL-Dye A, ffT-G2-AOL-Dye D, each comprising a 3′ AOM blocking group; (2) DNA polymerase Pol A; and (3) a glycine buffer.

In comparison, the incorporation mix used in the post incorporation labeling method included dark ffG, ffC-G2-AOL-TCO, ffA-G2-AOL-BL-NR550S0, ffA-G2-AOL-BL-Dye A, and ffT-G2-AOL-Dye D, each comprising a 3′ AOM blocking group. Post incorporation labeling mixture contains Dye A1-Tz in 10 mM pH 8 Tris buffer. High salt wash (1M NaCl in a standard post-cleavage wash solution that contains lipoic acid) was introduced after the incorporation step to remove excess polymerase, and also after the post incorporation labeling step to wash away the unreacted Dye A1-Tz. The post incorporation labeling mixture was introduced to incorporated ffNs and allowed to bind at 60° C. by flushing through the flow cell without incubation (2.5 s contact time).

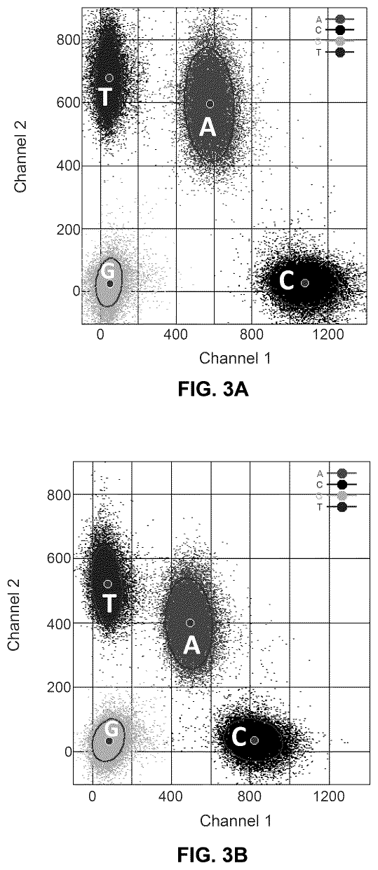

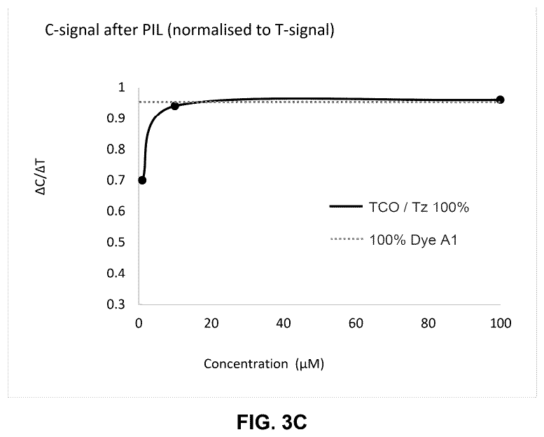

FIG. 3A is a scatterplot of cycle 6 of a blue/green two-channel SBS run conducted with a standard incorporation mixture containing fully labeled nucleotides in which the ffC is labeled with a blue coumarin dye A.

FIG. 3B is a scatterplot of cycle 12 of a blue/green two-channel SBS run conducted with an incorporation mixture containing a set of nucleotides including an unlabeled C nucleotide with trans-cyclooctene (TCO) moiety, and a post incorporation labeling mixture containing a blue coumarin dye A1 functionalized with a tetrazine (Tz) moiety (Dye A1-Tz) at 100 μM.

FIG. 3C is a line chart illustrating normalized C nucleotide signal (normalized to T signal) as a function of the concentration of a blue coumarin dye (A1-Tz) in the post incorporation labeling mixture, as compared to the normalized C nucleotide signal of an incorporation mixture containing ffC labeled with coumarin dye A.

### Example 8. Sequencing by Synthesis (SBS) Experiment on Illumina MiSeq® Instrument

In this experiment, two SBS runs were conducted on Illumina MiSeq® instrument with blue and green LED operating at 460 nm and 532 nm. Images were taken simultaneously through collection channels which are in blue (472-520 nm) and green (540-640 nm).

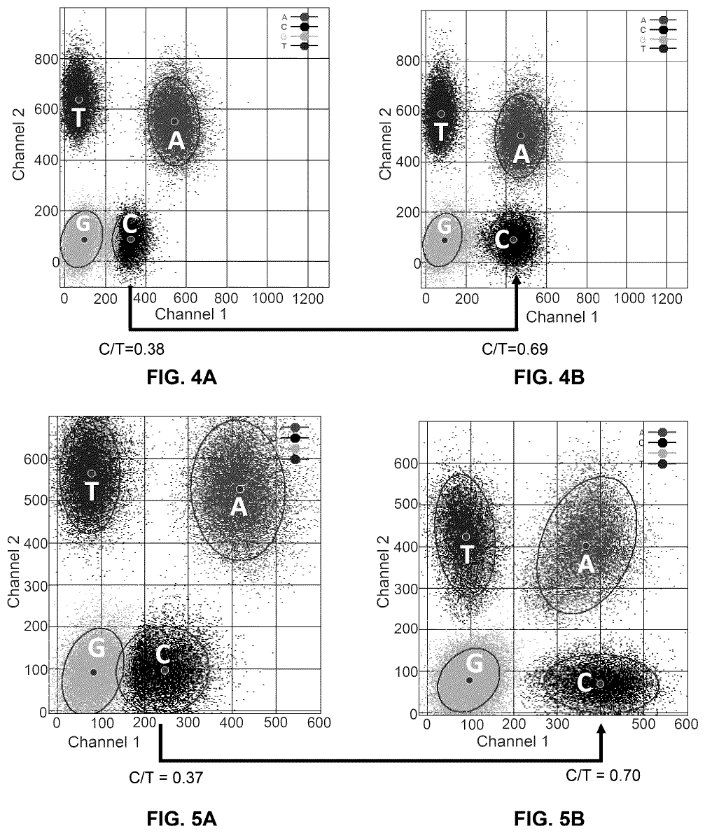

For scatterplot of FIG. 4A, the following incorporation mixture was used: (1) a set of nucleotides comprising dark ffG, DBCO-linked ffC (ffC-G2-AOL-DBCO), ffA-G2-AOL-BL-NR550S0+ffA-G2-AOL-BL-Dye A, ffT-G2-AOL-Dye D, each comprising a 3′ AOM blocking group; (2) DNA polymerase Pol A; and (3) a glycine buffer.

For scatterplot of FIG. 4B, the following incorporation mixture was used: dark ffG, DBCO-PEG₄-linked ffC (ffC-G2-AOL-PEG₄-DBCO), ffA-G2-AOL-BL-NR550S0+ffA-G2-AOL-BL-Dye A, ffT-G2-AOL-Dye D, each comprising a 3′ AOM blocking group.

For both SBS runs, the post incorporation reagent contains 1 mM Dye A1-PEG₃-N₃ in 10 mM pH 8 Tris buffer. The post incorporation mixture was introduced to incorporated ffNs and allowed to bind at 60° C. with 25 s incubation time.

FIG. 4A is a scatterplot of cycle 13 of a blue/green two-channel SBS run conducted with an incorporation mixture containing a set of nucleotides including ffC-G2-AOL-DBCO, and a post incorporation labeling mixture containing 1 mM Dye A1-PEG₃-N₃.

FIG. 4B is a scatterplot of cycle 28 of a blue/green two-channel SBS run conducted with an incorporation mixture containing a set of nucleotides including ffC-G2-AOL-PEG4-DBCO with extra polyethylene glycol (PEG) spacer, and a post incorporation labeling mixture containing 1 mM of Dye A1-PEG₃-N₃. For both SBS runs, the post incorporation labeling mixture was incubated at 60° C. for 25 s. It was observed that the extra PEG₄ linker to ffC-G2-AOL-DBCO improved accessibility of the labeling reagent (i.e., A1 functionalized with azido moiety) to react with the DBCO moiety, and resulted in improved clouds separation.

### Example 9. Sequencing by Synthesis (SBS) Experiment on Illumina MiSeq® Instrument

In this experiment, two SBS runs were conducted on Illumina MiSeq® instrument with blue and green LED operating at 460 nm and 532 nm. Images were taken simultaneously through collection channels which are in blue (472-520 nm) and green (540-640 nm).

For both scatterplots of FIG. 5A and FIG. 5B, the following incorporation mixture was used: (1) a set of nucleotides including dark ffG, ffC-G2-AOL-TCO, ffA-G2-AOL-BL-NR550S0+ffA-G2-AOL-BL-Dye A, ffT-G2-AOL-Dye D, each comprising a 3′ AOM blocking group; (2) DNA polymerase Pol A; and (3) a glycine buffer.

Post incorporation mixture containing 1 μM Dye A1-Tz in 10 mM pH 8 Tris buffer was used. The post incorporation mixture was introduced to incorporated ffNs and allowed to bind at 60° C. by flushing through the flow cell without incubation (2.5 s contact time). High salt wash (HSW) (1M NaCl in a standard post-cleavage wash solution that contains lipoic acid) was introduced after incorporation step and again after the post incorporation labeling to remove the excess unreacted Dye A1-Tz.

FIG. 5A is a scatterplot of a blue/green two-channel SBS run conducted with an incorporation mixture containing a set of nucleotides including ffC-G2-AOL-TCO, and a post incorporation labeling mixture containing 1 μM Dye A1-Tz. The post incorporation labeling mixture was introduced to the flowcell surface directly after the incorporation mixture was introduced.

FIG. 5B is a scatterplot of a blue/green two-channel SBS run conducted with the same incorporation mixture and the post-incorporation labeling mixture as described in FIG. 5A, in which a high salt buffer was used to flush through the flowcell surface after the incorporation step, and also after the post incorporation labeling step. It was observed that the use of high salt wash improved the scatterplot cloud separation.

### Example 10. Sequencing by Synthesis (SBS) Experiment on Illumina MiSeq® Instrument

In this experiment, three SBS runs were conducted on Illumina MiSeq® instrument with blue and green LED operating at 460 nm and 532 nm. Images were taken simultaneously through collection channels which are in blue (472-520 nm) and green (540-640 nm).

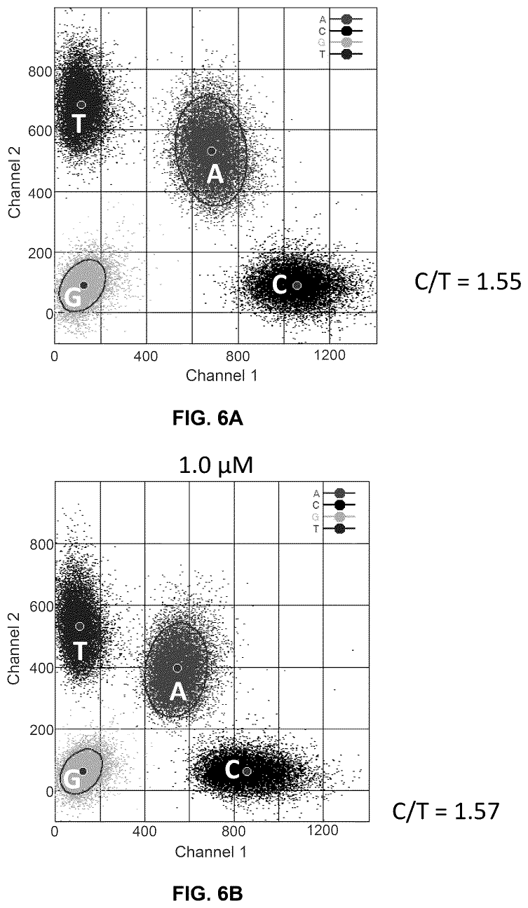

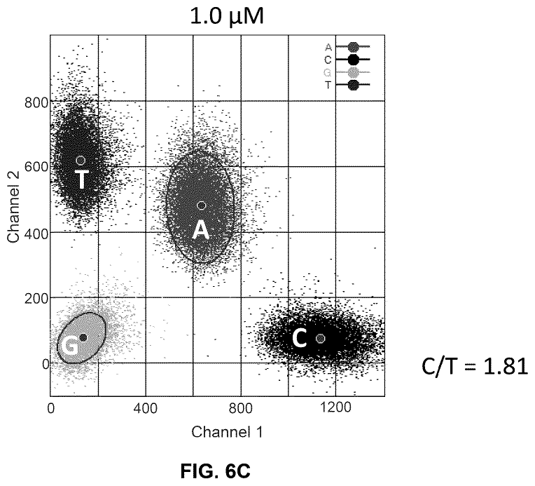

The standard incorporation mixture include: (1) a set of nucleotides comprising dark ffG, ffC-G2-AOL-Dye A, ffA-G2-AOL-BL-NR550S0, ffA-G2-AOL-BL-Dye A, ffT-G2-AOL-Dye D, each comprising a 3′ AOM blocking group; (2) DNA polymerase Pol A; and (3) a glycine buffer. The SBS run scatterplot at cycle 10 was illustrated in FIG. 6A.

In comparison, a second incorporation mix included dark ffG, ffC-G2-AOL-TCO, ffA-G2-AOL-BL-NR550S0+ffA-G2-AOL-BL-Dye A, ffT-G2-AOL-Dye D, each comprising a 3′ AOM blocking group. Post incorporation reagent contains 1 μM Dye A1-Tz in a buffer mix containing 0.75M NaCl and 0.06 mM citrate buffer at pH 7.5. High salt wash (1M NaCl in a standard post-cleavage wash solution that contains lipoic acid) was introduced after both the incorporation step and the post incorporation labeling step. The post incorporation mixture was introduced to incorporated ffNs and allowed to bind at 60° C. by flush through the flow bell without incubation (2.5 s contact time). The SBS run scatterplot at cycle 19 was illustrated in FIG. 6B.

A third incorporation mix included dark ffG, ffC-G2-AOL-AOL-TCO, ffA-G2-AOL-BL-NR550S0+ffA-G2-AOL-BL-Dye A, ffT-G2-AOL-Dye D, each with a 3′ AOM blocking group. Post incorporation reagent contains 1 μM Dye A1-Tz in a buffer mix containing 0.75M NaCl and 0.06 mM citrate buffer at pH 7.5. High salt wash (1M NaCl in a standard post-cleavage wash solution that contains lipoic acid) was introduced after both the incorporation step and the post incorporation labeling step The post incorporation mixture was introduced to the incorporated ffNs and allowed to bind at 60° C. by flushing through the flow cell without incubation (2.5 s contact time). The SBS run scatterplot at cycle 37 was illustrated in FIG. 6C. It was observed that extending the distance between ffC and the TCO moiety by introducing two AOL linkers resulted in increased C/T signal and improved scatterplot quality (comparing FIG. 6B to FIG. 6C).

### Example 11. Sequencing by Synthesis (SBS) Experiment on Illumina MiSeq® Instrument

In this experiment, three SBS runs were conducted on Illumina MiSeq® instrument with blue and green LED operating at 460 nm and 532 nm. Images were taken simultaneously through collection channels which are in blue (472-520 nm) and green (540-640 nm).

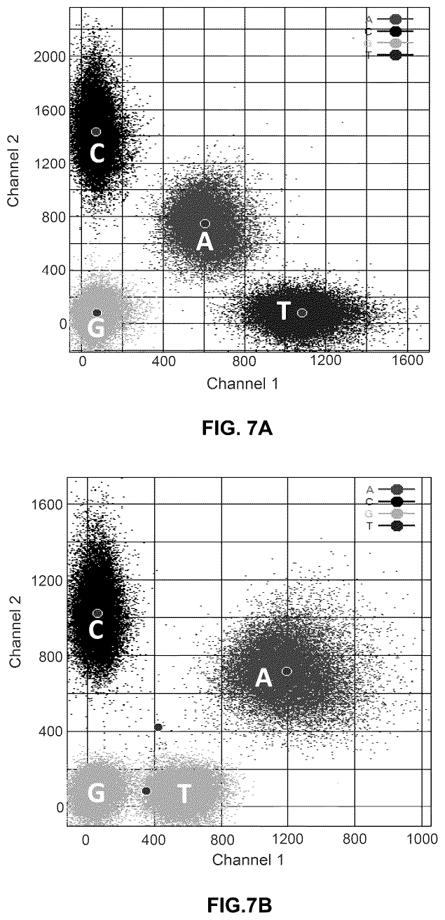

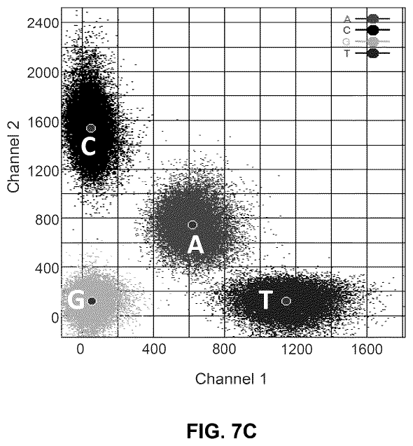

The standard incorporation mixture include: (1) a set of nucleotides comprising dark ffG, ffC-G2-AOL-Dye A, ffA-G2-AOL-BL-NR550S0, ffA-G2-AOL-BL-Dye A, ffT-G2-AOL-Dye D, each comprising a 3′ AOM blocking group; (2) DNA polymerase Pol A; and (3) a glycine buffer. The SBS run scatterplot at cycle 6 was illustrated in FIG. 7A.

A second incorporation mix included dark ffG, ffT-G2-AOL-DBCO, ffA-G2-AOL-TCO, ffC-G2-AOL-NR550S0+ffC-G2-AOL-Dye A, each comprising a 3′ AOM blocking group. Post incorporation reagents contain 5 μM NR550C4-Py-Tz and 500 μM Dye A1-PEG₃-N₃ in a buffer mix containing 0.75M NaCl and 0.06 mM citrate buffer at pH 7.5. High salt wash (1M NaCl in a standard post-cleavage wash solution that contains lipoic acid) was introduced after the incorporation step and the post incorporation labeling step. The post incorporation mixture was introduced to incorporated ffNs and allowed to bind at 60° C. by flushing through the flow cell without incubation (2.5 s contact time) and also with 20 s incubation time. The SBS run scatterplot at cycle 9 without incubation was illustrated in FIG. 7B and the SBS run scatterplot at cycle 11 with 20 s incubation was illustrated in FIG. 7C.

Furthermore, SBS runs using a fully unlabeled ffN set including dark G, ffA-TCO, ffT-DBCO, ffC-TCO and ffC-DBCO in conjunction with a set of post incorporation reagents containing 5 μM NR550C4-Py-Tz and 500 μM Dye A1-PEG₃-N₃ were also performed, demonstrating the mutual biorthogonality of TCO/Tz and DBCO/N₃ pair on flowcell.

1. A method of determining the sequences of a plurality of different target polynucleotides, comprising:
(a) contacting a solid support with a solution comprising sequencing primers under hybridization conditions, wherein the solid support comprises a plurality of different target polynucleotides immobilized thereon; and the sequencing primers are complementary to at least a portion of the target polynucleotides;
(b) contacting the solid support with an incorporation mixture comprising DNA polymerase and one or more of four different types of nucleotides under conditions suitable for DNA polymerase-mediated primer extension, and incorporating one type of nucleotides into the sequencing primers to produce extended copy polynucleotides; wherein
each of the four types of nucleotides comprises a 3′ blocking group; and
the incorporation mixture comprises a first type of unlabeled nucleotide having a first functional moiety covalently attached to the first type of unlabeled nucleotide;
(c) contacting the extended copy polynucleotides with a first labeling reagent comprising a first detectable label and a first reactive moiety that reacts specifically with the first functional moiety of the first type of unlabeled nucleotides to form covalent bonding;
(d) imaging the solid support and performing one or more fluorescent measurements of the extended copy polynucleotides; and
(e) removing the 3′ blocking group of the incorporated nucleotides.

2. The method of claim 1, wherein the first functional moiety is covalently attached to the nucleobase of the first type of unlabeled nucleotide via a cleavable linker.

3. (canceled)

4. The method of claim 1, wherein the first reactive moiety of the first labeling reagent forms covalent bonding with the first functional moiety of the first type of unlabeled nucleotides via a biorthogonal reaction selected from the group consisting of a [3+2] dipolar cycloaddition, a Diels-Alder cycloaddition, a [4+1] cycloaddition, a phosphine ligation, and condensation with 2-acylphenyl boronic acid.

5. (canceled)

6. The method of claim 1, wherein one of the first functional moiety and the first reactive moiety comprises alkynyl, unsubstituted or substituted dibenzocyclooctyne moiety (DBCO) 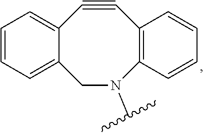 [SMILES: *N1Cc2ccccc2C#Cc2ccccc21]
unsubstituted or substituted bicyclo[6.1.0] nonyne (BCN) moiety, unsubstituted or substituted norbornene moiety, unsubstituted or substituted transcyclooctene (TCO) moiety, primary isonitrile, tertiary isonitrile, vinyl boronic acid, 2-acylphenyl boronic acid, phosphine methyl ester, phosphine alcohol, phosphine amine, or phosphine thiol, and the other one of the first functional moiety and the first reactive moiety comprises azido, sydnone, imino sydnone, nitrone, phenyl tetrazine, pyrimidyl tetrazine, methyl tetrazine, pyridyl tetrazine, t-butyl tetrazine, triazine, cyclopropenone, cyclopropenium ion, DTO, chloro-oxime, or amino hydrazide.

7. The method of claim 6, wherein one of the first functional moiety and the first reactive moiety comprises norbornene, DBCO, BCN, or optionally substituted triphenylphosphine moiety, and the other one of the first functional moiety and the first reactive moiety comprises azido.

8. The method of claim 7, wherein the first functional moiety comprises norbornene, DBCO, or BCN, and the first reactive moiety comprises azido.

9. (canceled)

10. The method of claim 6, wherein one of the first functional moiety and the first reactive moiety comprises TCO, and the other one of the first functional moiety and the first reactive moiety comprises a substituted tetrazine.

11. The method of claim 6, wherein one of the first functional moiety and the first reactive moiety comprises an amino hydrazide moiety, and the other one of the first functional moiety and the first reactive moiety comprises a 2-acylphenyl boronic acid moiety.

12. The method of claim 1, wherein the incorporation mixture comprises a second type of labeled nucleotide, and a third type of labeled nucleotide.

13. The method of claim 1, wherein the incorporation mixture comprises a second type of unlabeled nucleotide having a second functional moiety covalently attached to the second type of unlabeled nucleotide, and a third type of labeled nucleotide.

14. The method of claim 1, wherein the incorporation mixture comprises a second type of unlabeled nucleotide having a second functional moiety covalently attached to the second type of unlabeled nucleotide, and a mixture of a third type of unlabeled nucleotide having a first functional moiety covalently attached to the third type of unlabeled nucleotide and a third type of unlabeled nucleotide having a second functional moiety covalently attached to the third type of unlabeled nucleotide.

15. The method of claim 14, wherein step (c) further comprises contacting the extended copy polynucleotides with a second labeling reagent comprising a second detectable label and a second reactive moiety that reacts specifically with the second functional moiety to form covalent bonding.

16. The method of claim 1, wherein the incorporation mixture comprises a second type of unlabeled nucleotide having a second functional moiety covalently attached to the second type of unlabeled nucleotide, and a third type of unlabeled nucleotide having a third functional moiety covalently attached to the third type of unlabeled nucleotide.

17. The method of claim 16, wherein step (c) further comprises contacting the extended copy polynucleotides with a second labeling reagent comprising a second detectable label and a second reactive moiety that reacts specifically with the second functional moiety of the second type of unlabeled nucleotides to form covalent bonding, and a third labeling reagent comprising a third detectable label and a third reactive moiety that reacts specifically with the third functional moiety of the third type of unlabeled nucleotides to form covalent bonding.

18. The method of claim 17, wherein the incorporation mixture comprises a fourth type of unlabeled nucleotide, wherein the fourth type of unlabeled nucleotide is not capable of reacting with any of the labeling reagents.

19. The method of claim 1, wherein step (e) also removes the detectable labels of the incorporated nucleotides.

20. (canceled)

21. The method of claim 19, further comprising (f) washing the solid support with an aqueous wash solution, wherein steps (b) to (f) are repeated at least 50, 100, 150, 200, 250, 300, 350, 400, 450, or 500 cycles to determine the target polynucleotide sequences.

22. (canceled)

23. (canceled)

24. The method of claim 1, wherein the first labeling reagent is contacted with the extended copy polynucleotides in step (c) by flushing the first labeling reagent through the extended copy polynucleotides.

25. The method of claim 1, wherein after step (b) the extended copy polynucleotides on the solid support are washed with a high salt buffer solution prior to contacting with the first labeling reagent.

26. The method of claim 1, wherein after step (c) the extended copy polynucleotides on the solid support are washed with a buffer solution prior to imaging the solid support.

27. The method of claim 1, wherein the method is performed on an automated sequencing instrument comprising two light sources operating at two different wavelengths.

28. The method of claim 27, wherein one light source has a wavelength of about 450 nm to about 460 nm, and the other light source has a wavelength of about 520 nm to about 540 nm.

29. A kit for sequencing application, comprising:
an incorporation mixture composition comprising one or more of four different types of nucleotides each comprising a 3′ blocking group, wherein a first type of unlabeled nucleotide having a first functional moiety covalently attached to the first type of unlabeled nucleotide; and
a first labeling reagent comprising a first detectable label and a first reactive moiety that is capable of reacting specifically with the first functional moiety of the first type of unlabeled nucleotides to form covalent bonding.

30.-50. (canceled)

51. A system for nucleic acid sequencing, comprising a plurality of chambers, wherein one of the chambers contains the kit of claim 29.
